(19) United States
(12) Patent Application Publication
(10) Pub. No.: US 2007/0102280 A1
(43) Pub. Date: May 10, 2007

(54) Air supply apparatus

(76) Inventors: C. Eric Hunter, Jefferson, NC (US); Jocelyn L. Hunter, Jefferson, NC (US); Bernard L. Ballou JR., Raleigh, NC (US); John H. Hebrank, Durham, NC (US); Laurie E. McNeil, Chapel Hill, NC (US)

Correspondence Address: Richard S. Faust, Suite 204, 8384 Six Forks Road, Raleigh, NC, 27615, US

(21) Appl. No.: 11/434,552

(22) Filed: May 15, 2006

Related U.S. Application Data
(63) Continuation-in-part of application No. 11/268,936, filed on Nov. 8, 2005.
(63) Continuation-in-part of application No. 11/317,045, filed on Dec. 23, 2005.
(63) Continuation-in-part of application No. 11/412,231, filed on Apr. 26, 2006, now abandoned.
(60) Provisional application No. 60/796,368, filed on May 1, 2006.

(51) Int. Cl. C07C 1/00; B01J 19/12

(52) U.S. Cl. 204/157.15; 422/186.3

(57) ABSTRACT

In an air sterilization system that includes a UV kill chamber for sterilizing air that is to be supplied to users, the effectiveness of killing or neutralizing pathogens is increased by including not only a UV light source of a certain intensity but also including a particle filter and providing short duration high intensity UV radiation. In the case of a user specific system that includes a face mask to supply air to a specific user, exhaled air from the face mask may be sterilized as well, either by using the same kill chamber or by using a separate kill chamber.

This is a continuation-in-part of U.S. application Ser. No. 11/268,936 to Charles Eric Hunter, filed Nov. 8, 2005; of Ser. No. 11/317,045 to Charles Eric Hunter filed Dec. 23, 2005; of the patent application Ser. No. 11/412,231 entitled “Air Supply Apparatus” filed Apr. 26, 2006, and of provisional patent application 60/796,368 entitled “Air Supply Apparatus” filed May 1, 2006.

### FIELD OF THE INVENTION

The invention relates to an air supply system and applications of the air supply system to kill airborne organisms such as viruses, bacteria and fungi, also referred to as organic material, pathogens or biological contaminants using ultraviolet (UV) radiation. For purposes of this application the term “killing” also includes any DNA or RNA destruction.

### BACKGROUND OF THE INVENTION

In order to provide an effective sterilization respirator based on UV sterilization the present application recognizes the need to take into account air consumption rates by the user. The present invention therefore takes into consideration the peak respiration of a typical person under certain working conditions and factors in a maximum flow through the respirator. By way of example, the present invention deals with the design of the respirator that focuses on providing a safe supply of air for persons working in a pandemic environment performing moderate exercise. Moderate or light exercise is defined by NIOSH as work not exceeding 50 watts. This level of activity equates to the average adult walking at a rate of three miles per hour. NIOSH sets the peak respiration at 85 SLM under these conditions where the air consumption in minute-liters is 25.

The embodiments discussed below target essential workers and their families that will be performing only moderate exercise, not first responders or members of our military that perform exercise at levels of 150 watts and greater. It will, however, be appreciated that the approach described is scalable to high-end applications or any other applications.

The specifications for the respirator apparatus targeting the essential worker and their families are:

Maximum Flow—220 SLM (this is through the filter to the mask)

Peak Respiration—90 SLM (1.5 liters per Second) {with S<10 E−11}

Power Consumption—7 watts

Battery Charge—8 hours (based on a degraded 70 watt-hour battery pack)

Weight—2.5 lbs. (battery and UV chamber weight is 1.5 lbs.)

The most complete attempt to model the elimination of active airborne pathogens using UVC light is Mathematical Modeling of Ultraviolet Germicidal Irradiation for Air Disinfection by Kowalski et al, in the Journal: Quantitative Microbiology 2, 249-270, 2000. The paper outlines a classical approach to dealing with pathogen population decay defined by the equation S=e(−kIt). Where S is the fraction of the pathogen population that survives exposure, I is the intensity in microwatts per square cm, k is the standard rate constant for a particular pathogen expressed in square cm per micro joule and t is the exposure time in seconds.

As outlined by Kowalski et al, research with 8 known pathogens, including three viruses, has shown a secondary population that survives after the initial exposure. This population is dealt with using the classical approach by assigning a second rate constant k2 and adding the decay of this population to the first using the same equation S=e(−k2It). Information regarding the values for K2 is limited, only being available for 8 pathogens. Reasons for a secondary survival population can be ascribed to one or more of several possibilities, including 1) higher resistance to UVC 2) clustering of pathogens and 3) non-optimum chamber design where intensity (photon flux) is wildly uneven. (Intensity being high nearest to the lamp and much lower elsewhere). In the past, dose studies were typically performed by projecting UVC light onto pathogens on a surface. It is therefore likely that under these conditions reasons 1 and/or 2 are primarily responsible for the secondary survival population of pathogens.

The third reason, however, suggests that actual results in UVC systems to date have been poor; since all known systems have utilized a design where air flows past a round lamp having a photon flux that varies dramatically based on the lamp radius and the distance from the lamp. In fact, some literature, incorrectly teaches that intensity drops as a square from the distance to the lamp, not even considering the lamp radius {as the radius approaches zero the ratio of X1 (the intensity beside the lamp)/X2 (the intensity at some distance away from the lamp) goes to infinity}. More sophisticated attempts to model the intensity field (such as Kowalski et al) deal with more than 15 variables many of which are difficult to measure or predict, and even these models show a wide variation in intensity with current chamber designs.

Most importantly, prior art systems have not provided an evaluation or determination of the success of air sterilization systems and have made no attempts at measuring low pathogen concentrations. The fact that these systems have dramatic variations in effectiveness as shown both in demonstations and through the use of models means that secondary effects such as k2 that were measured on a planar surface have not been addressed in prior art systems.

The present invention seeks to address some of these issues by making use of a sterilization or kill chamber that includes a pump, a fan, or a blower in which the flow rate is contolled. In order to address the secondary survival of pathogens due to uneven UV intensity, the present invention further proposes providing a high intensity radiation zone.

The use of pumps, fans, and blowers to move fluids is known. For instance air in rooms is commonly circulated by making use of ceiling mounted or standing fans. These typically include a number of settings for manually adjusting the fan speed to suit the user's preferences. However, in the case of pumps, blowers or fans mounted in a housing or conduit in order to move air through the housing or conduit, no known system automatically adjusts power to the pump, blower or fan or adjust shutters or other mechanisms such as a butterfly valves in order to achieve constant flow or constant pressure as external factors vary and therefore seek to impact the flow rate or air pressure. The present invention proposes a system in which flow rate or air pressure in the system is controlled to keep flow rate or pressure substantially constant.

In the field of air purification much work has been done to filter out particles, e.g., filters in air duct systems found in many forced air home heating units. Filters are also used to filter out harmful particles in face masks as is discussed below. In the case of biological contaminants, considerable work has also been done in sterilizing water using mercury vapor lamps, and the use of vacuum UV sources to kill biological contaminants in air has also been considered. For instance, Brais, U.S. Pat. No. 5,833,740 discloses a chemical air purification and biological purification using UV sources, and making use of a turbulence generator mounted within the housing. Air purification by means of UV is also discussed in Kaura, U.S. Pat. No. 6,623,544B1. In this patent the air is treated with mechanical filters (including electrostatic filters), ionization of energetic ions, and UV light radiation. The PAPR made by 3M, on the other hand, comprises an air purifier making use of chemicals to kill biological pathogens.

Showdeen, et al., U.S. Pat. No. 5,446,289 also discusses the sterilization of articles by means of UV lamps mounted in a chamber.

However, the prior art systems making use of UV sources to kill biological contaminants in air do not consider controlling the flow rate past the UV radiation source in order to control the UV dosage to which the contaminants are exposed or controlling the pressure in a kill or sterilization chamber. More particularly, they do not consider moving the air past a UV source using a pump, fan or blower and adjusting the flow rate of the air by adjusting power to the pump, fan or blower. Thus the prior art also does not consider power saving, by automatically adjusting power to the pump, fan or blower in response to changing demands, which is particularly important in portable devices.

Furthermore, the prior art systems do not ensure that biological contaminants passing through a kill or sterilization chamber or through a sterilization zone, e.g., a UV radiation zone provided in an air duct system of a house, ship or aircraft, receive an adequate amount of radiation to render them harmless. Nor do they optimize power usage in portable devices, or consider the possible harmful byproducts of UV radiation, such as ozone and carbon monoxide.

Also there is no art that teaches actively destroying biological contaminants in a face mask assembly using ultraviolet radiation. When it comes to the field of face masks, masks with various types of filters are commonly known. Wadsworth, et al., U.S. patent application publication 2005/0079379 A1, for instance, describes an improvement on such a face mask using a two-layer or multi-ply barrier fabric having at least one barrier fabric layer which is impermeable to liquids but allows moisture vapor to pass through the micropores and in which the layers may contain an antimicrobial agent. Kirollos, et al., U.S. patent application publication 2004/0223876, in turn, describes exposure protection equipment such as a respiratory protection device, which includes a detector for indicating the presence of a target substance.

While Wen, U.S. patent application publication 2003/0111075 A1 describes a gas mask that kills bacteria, it does so using chemical agents. Wen makes use of a filtration apparatus containing an active stage and a passive stage, the active stage containing at least one chemical agent to kill ambient bacteria and viruses.

The present invention seeks to address these issues and seeks to provide not only sterile air to the user by means of a portable face-mask arrangement but also proposes sterilization of air exhaled by the user.

### SUMMARY OF THE INVENTION

According to the invention there is provided an air sterilization system, comprising a UV light source for providing UV light of a predefined intensity, a blower having an input and an output, a filter, e.g., a HEPA filter mounted at the input or output of the blower, the air pressure or air flow rate of the air supply being automatically adjusted to account for changes in the demand the system further comprising means for radiating pathogens with high intensity UV light in a high intensity zone, wherein the high intensity light is of a higher intensity than the predefined intensity. The high intensity light may be created by a UV beam magnifier such as a UV lens or by a separate high power light source. It will be appreciated that providing a high intensity zone with high intensity light exposure is applicable to both user specific devices that make use of face masks, as well as to multi-user systems such as air duct sterilization systems.

Further, according to the invention there is provided an air sterilization system for providing a sterile air supply to a face mask, comprising a face mask having an air input and an air output, a kill chamber that includes a housing having an input for receiving air from the atmosphere and an output connected to the input of the face mask, a UV light source, a pump, fan or blower for generating an air stream, and a particle filter, e.g. a HEPA filter mounted on the housing, wherein air pressure or air flow rate of the air supply is automatically adjusted to account for changes in the demand, and wherein the system includes one or both of a UV beam magnifier and a second input to the housing connected to the output from the face mask. The system may measure the flow rate of the air stream or air pressure using a sensor and use the sensor signal to control the flow rate of said air stream or the air pressure in the air stream. The flow rate or pressure may be controlled by controlling power to the pump, fan or blower or may be controlled by adjusting a manually controlled or an electronically controlled valve mounted in the housing or conduit or mounted upstream or downstream of the housing or conduit, or by adjusting both the pump, fan or blower as well as such a valve. In particular, flow rate may be adjusted to provide for substantially constant flow, or pressure may be adjusted to provide a substantially constant pressure. The valve may include a hole to bleed air through the valve or may be adapted to always be at least partially open to ensure a slight positive pressure. The system may be a portable system in which power to the pump, fan or blower and any electronically controlled valve are powered by at least one battery. Changes in pressure caused by the inhaling and exhaling of the user may be adjusted for to provide a constant air flow rate or constant air pressure system. In particular, a pressure sensor mounted in the housing or conduit, or in the mask may be used to provide a pressure signal for use in adjusting power to the pump, fan or blower and/or to control an electronically controlled valve, in order to provide air to the user on demand, thereby providing a positive pressure in the mask while avoiding excessive pressure build-up during exhaling or low exertion by the user, while ensuring sufficient air flow during inhaling irrespective of the level of exertion of the user. Thus, in a constant pressure system of the invention, one embodiment provides for adjusting a valve to accommodate pressure changes due to inhaling and exhaling by a user (since the system of the invention seeks to maintain constant pressure). As the valve changes, flow rate changes, which impacts how hard the pump, fan or blower has to work (since the air has to be accelerated from zero on the upstream side of the pump, fan, or blower, to the particular flow rate needed on the downstream side of the pump, fan, or blower.) In cases where power to a blower or fan is adjusted, preferably a fan or blower designed to have low inertia is used e.g. through the use of graphite components and further providing means for quickly stopping the fan when air flow is not required. The stopping may be achieved through the use of an electrically activated micro brake. The fan or blower may make use of multiple motors of the same or different power that can be individually activated to optimize power consumption by powering only a chosen number of motors or a motor of the chosen power for a desired flow rate.

The pressure sensor may be located near or on the mask to limit errors due to pressure drops along the delivery tube. The sensor can provide a voltage or current output. Preferably the signal is a mixed signal device wherein a small voltage signal is digitized to ensure accuracy of the transmitted signal. Preferably multiple sensors are used that can be averaged or where high and low values are thrown out to ensure repeatability and stability of the signal. The sensors may be temperature controlled to avoid errors due to changes in ambient temperature. The system may also be used in conjunction with an ultraviolet (UV) light source to kill or destroy biological pathogens. The nature of the filter may be chosen to limit clusters or clumps of the particular biological pathogen(s) that the UV light source is intended to kill or destroy. Typically a filter capable of filtering 0.1 μm diameter or smaller pathogens is used. In order to address biological contaminants with a higher resistance to UV radiation (secondary survival rates of pathogens), a high intensity zone may be defined at the input or output to the housing or conduit or any other location in the housing or upstream or downstream of the housing and may include a small hole or passage e.g., a 0.3 cm² hole through which the air is passed and which defines a high intensity zone. The UV beam magnifier create the high intensity radiation by focusing the UV beam on the high intensity zone e.g. on the 0.3 cm² hole. Thus the means for radiating pathogens with high intensity UV light may comprise a beam magnifier, which typically includes a lens made of high transmissivity material such as silicon dioxide. The high intensity zone may include a highly reflective cylinder extending from the hole to define a channel to ensure sufficient exposure time to the air passing through the high intensity zone (or to ensure exposure to a pulse in the case of a flash lamp, discussed below). Instead of a UV mercury vapor lamp, a UV laser or a flash lamp (e.g., xenon or xenon-mercury flash lamp produced by Perkin Elmer such as the RSL3100) producing a high intensity burst of UV light or other energy source may be used. In such a case, the beam magnifier may in some embodiments be used with the UV laser or flash lamp. The system is typically a portable system and may be powered by one or more replaceable or rechargeable batteries, e.g., lithium ion batteries. Air exhaled by the user may be sterilized by channeling the exhaled air to the second input of the housing or may be sterilized by supplying the exhaled air to a separate kill chamber.

Still further, according to the invention, there is provided a method of reducing pathogens in an air stream, comprising exposing the air stream to a first intensity UV radiation for a first predefined period of time, and exposing the air stream to an elevated intensity of UV radiation that is higher than said first intensity. The elevated intensity may include a range of elevated intensities and exposure to the elevated intensity may be for a duration that is less than the first predefined period of time, and may include the time during which the air stream passes through a high intensity zone. UV radiation for a first predefined period of time may be defined by the time that it takes the air stream to pass through a certain region, e.g., through a housing. The elevated intensity may be provided by a beam magnifier, e.g., a UV lens. The high intensity zone may comprise a channel through which the air stream is forced to pass or may comprise part of the housing.

Still further, according to the invention, there is provided a method of providing protection against airborne pathogens, comprising (a) providing a face mask for channeling air to a user, and (b) sterilizing the air that is channeled to the user, using UV radiation. The method may include sterilizing the air exhaled by said user. The method may include controlling the flow rate or pressure of air channeled to the user. The peressure may be controlled to maintain substantially constant pressure during inhaling and exhaling by the user and during changes in exertion by the user. The air stream provided by the blower/fan/ pump or the pressure may be controlled by controlling at least one of a flapper valve and the blower, fan or pump.

### BRIEF DESCRIPTION OF THE DRAWINGS

FIG. 1 shows a simplified representation of one embodiment of a portable sterilization apparatus of the invention;

FIG. 2 shows another embodiment of part of a sterilization apparatus of the invention;

FIG. 3 shows yet another embodiment of part of a sterilization apparatus of the invention;

FIG. 4 shows yet another embodiment of part of a sterilization apparatus of the invention;

FIG. 5 shows a user wearing yet another embodiment of a portable sterilization apparatus of the invention;

FIG. 6 shows a longitudinal section through part of another embodiment of a sterilization apparatus of the invention;

FIG. 7 is a top view of the embodiment of FIG. 6;

FIG. 8 shows a cross section through the apparatus of FIG. 6 along the line A-A;

FIG. 9 shows a side view of the apparatus of FIG. 6 connected to a mask shown in three dimensions;

FIG. 10 is a three dimensional view of another embodiment of a mask assembly of the invention;

FIG. 11 is a three dimensional view of another embodiment of a mask assembly of the invention;

FIG. 12 shows a block diagram of one embodiment of the electronic circuitry of the invention;

FIG. 13 is a three dimensional view of another embodiment of a kill chamber of the invention;

FIG. 14 is section through part of the embodiment of FIG. 13;

FIG. 15 is a section through another part of the embodiment of FIG. 13;

FIG. 16 shows one embodiment of a kill chamber and power supply in duplicate, housed in a fanny pack,

FIG. 17 shows another embodiment of a kill chamber and power supply housed in a fanny pack,

FIG. 18 is a section through part of another embodiment of a kill chamber of the invention,

FIG. 19 is a section through part of yet another embodiment of a kill chamber of the invention,

FIG. 20 is a simplified cross section through one embodiment of a kill chamber of the invention,

FIG. 21 is a simplified cross section through another embodiment of a kill chamber of the invention,

FIG. 22 is a cross section through part of another embodiment of a kill chamber of the invention,

FIG. 23 is a simplified cross section through yet another embodiment of a kill chamber of the invention showing the use of a fan or blower,

FIG. 24 is a simplified cross section through part of yet another embodiment of a kill chamber of the invention showing the use of a fan or blower,

FIG. 25 is a simplified cross section through part of yet another embodiment of a kill chamber of the invention showing the use of a fan or blower,

FIG. 26, shows a method of making a housing for a kill chamber,

FIG. 27 shows another method of making a housing for a kill chamber,

FIG. 28 shows an embodiment of a pair of gloves of the invention,

FIG. 29 shows another embodiment of a pair of gloves of the invention packaged in a sealed plastic bag,

FIG. 30 shows a pad for removing gloves used with the apparatus of the invention,

FIG. 31 shows a three dimensional view of a decontamination chamber of the invention,

FIG. 32 shows an embodiment of the side and back panels of the chamber of FIG. 31,

FIG. 33 shows an embodiment of the upper and lower panels of the chamber of FIG. 31,

FIG. 34 shows one embodiment of a support arrangement for supporting the apparatus and clothing items inside the chamber of FIG. 31,

FIG. 35 shows a longitudinal section through part of yet another embodiment of a sterilization apparatus of the invention,

FIG. 36 shows a longitudinal section through part of yet another embodiment of a sterilization apparatus of the invention,

FIG. 37 shows a longitudinal section through part of yet another embodiment of a sterilization apparatus of the invention, and

FIGS. 38-44 show longitudinal sections through parts of three other embodiments of a sterilization apparatus of the invention

### DETAILED DESCRIPTION OF THE INVENTION

As mentioned above, the present invention defines an air supply system providing an air stream, the system including a filter and a means for moving the air, e.g., a pump, fan, or blower, as well as means for controlling either the flow rate of the air stream or the air pressure. In contrast, prior art devices make use of constant high flow rates which prevents use of good HEPA filters due to the large pressure drop. Also, they produce a large positive pressure causing constant expulsion of air and are therefore typically used with visor-like masks that allow air to freely pass from the mask. Since they are not on-demand systems they will potentially expose the user to more contaminated air. The present invention, on the other hand, makes use of a controlled air flow system to avoid these drawbacks. The embodiments of the present application, further include means for killing or destroying organic contaminants in the air stream by radiating the air stream with UV radiation.

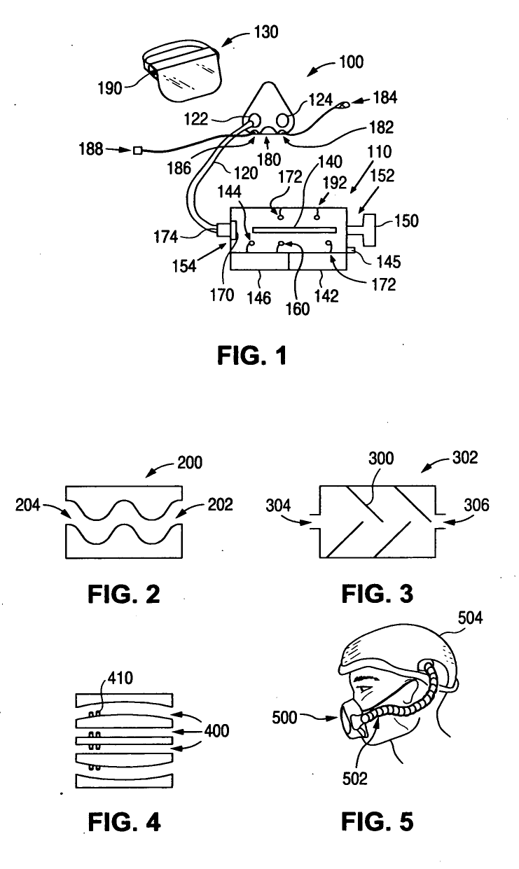

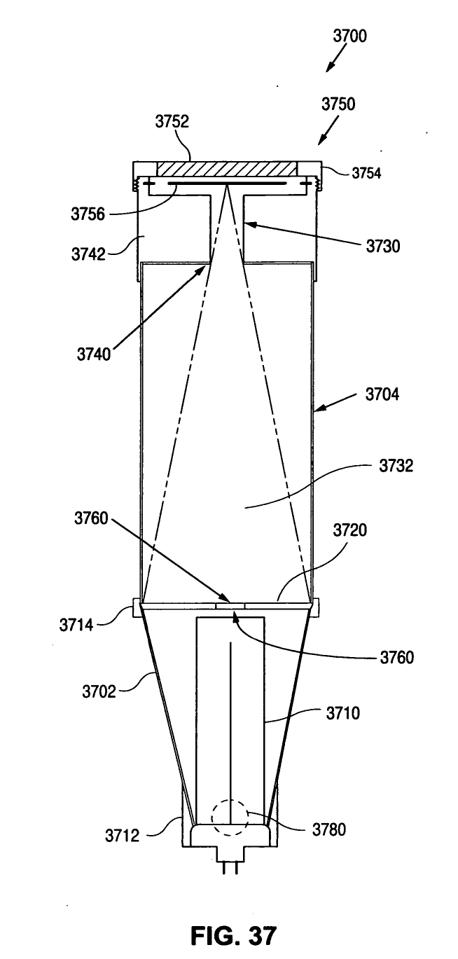

For ease of understanding some of the concepts and elements that will be discussed with respect to FIGS. 37-40, the present invention includes embodiments and description from earlier filed applications that the present application claims priority from. One such previously discussed embodiment of a portable air sterilization apparatus of the invention is shown in FIG. 1, which shows a face mask 100 connected to a kill chamber 110 by means of a flexible delivery tube 120. The face mask 100 includes a one-way intake valve 122 and a one-way exhaust valve 124. The face mask 100 fits over a person's nose and mouth with the exhaust valve 124 sending the exhaled air into the atmosphere. The intake valve 122 allows the person to inhale sterilized air. The one-way valves 122, 124 ensure that the person breathes sterilized air while eliminating the used air to the atmosphere. As will be discussed in greater detail below with respect to FIGS. 37-40, the embodiments of the present invention include the possibility of radiating the exhaled or used air with UV radiation instead of simply venting it to the atmosphere. Valves 122, 124 may be simple flapper valves, over center flapper valves, or electrically actuated valves. In one embodiment, the valve open area was chosen correspond approximately to the cross-section of a human trachea (about 3-5 cm²). The delivery tube 120 which is preferably made of a flexible material is chosen to have a similar cross-section (3-5 cm²). In a preferred embodiment, the mask 100, valves 122, 124, and delivery tube 120 are designed to be removable from the kill chamber or sterilizer chamber 110 to facilitate washing, and are preferably made of a dishwasher safe material. By providing for quick release connectors or otherwise providing connectors that allow the kill chamber, delivery tube or hose, and face mask to be readily separated from each other, the various parts allow for easy exchange of worn out parts or use of components from another apparatus. In one embodiment, the apparatus may include eye protection such as glasses or goggles, or a flip-down transparent visor as indicated by reference numeral 130. The visor 130 of this embodiment includes a heads-up display and a receiver 190 for receiving external feed for displaying information on the display 130. The receiver 190 may be a wireless receiver e.g. a WiFi receiver for receiving wireless Internet feed or cached content information feeds. In the embodiment shown, an air pump 170 is included in the chamber 110 to provide a positive pressure within the mask 100 thereby ensuring that the surrounding air is not inadvertently drawn into the mask 100 along its sides where it abuts the user's face. The pump 170 also serves to ease the inhaling process by providing an air flow toward the mask 100. One such pump is a diaphragm pump, e.g. 7010/−2.2N DC 12V and 24V produced by Rietschle Thomas of Sheboygan, Wis. Instead of pushing air directly to the mask, the pump, in another embodiment my supply a supply tank which then feeds the face mask via an appropriate regulator at the mask or tank.

In this embodiment, the sterilizer or kill chamber 110 has an internal volume corresponding approximately to one human breath of an adult under moderate exertion. (The typical breath of a resting adult is about 0.5 liter and under moderate exertion volumes will typically increase to 1 and 1.5 liters for a typical adult.) However, as is discussed in greater detail below, flow rate through the chamber is monitored to ensure that larger breaths and rapid breathing may be taken into consideration. The invention is not, however, limited to such an arrangement. As is discussed below, in other embodiments the chamber volume is specifically chosen to be smaller than an average breath of a typical user of the apparatus. In the present embodiment the kill chamber 110 is tubular in shape with a diameter of approximately three inches (3″) and six to eight inches (6-8″) in length. A UV light source 140 is mounted in the chamber 110. In one embodiment the UV light source is a mercury vapor lamp mounted by means of brackets (not shown) to extend substantially along the center of the chamber. In the embodiments using a mercury vapor lamp as the UV light source, the lamp is protected in a quartz sleeve to reduce the likelihood of breakage. Also, a sensor 172 is included to monitor the output of the mercury vapor lamp and close a valve 174 to the mask 100 if the lamp stops radiating. This will ensure that no noxious gases from the lamp, nor untreated air is passed into the user's lungs. Preferably multiple UV sensors are includes since they tend to degrade over time. Therefore multiple sensors to monitor the amount of UV radiation are beneficial in ensuring that the UV source produces sufficient UV. The sensor 172 can be a photodetector made from AlGaN, SiC, AlN, GaN, InGaN, AlInGaN, GaAs, Si, or AlN:SiC alloys. Preferably the photodetectors are filtered to cut out wavelengths that are not cut out by the earth's ozone layer (currently 280 nm and above), either by means of an on-chip deposited filter, e.g. doped SiO₂, or by means of a separate filter such as those sold by the company Schott in Mainz, Germany. The filtering ensures avoiding incorrect readings caused by extraneous UV interference. Preferably additional photodetectors clipped at 400 nm are included that measure light above 400 nm (visible light) to ensure that there is no light leakage into the chamber. This ensures that there are no gaps in the chamber that would allow UV light to escape. In the case of a mercury vapor lamp as UV light source, instead of monitoring UV light using a photodetector, the sensor 172 can instead simply be a current sensor for monitoring current through the lamp.

It will be appreciated that the dimensions of the chamber 110 may vary depending on the nature, size, and configuration of the UV light source. The inner surface of the chamber 110, in this embodiment, is coated with a UV reflective coating, such as aluminum with a silicon dioxide protective coating so that radiation from the UV light source 140 will pass through the air in the chamber multiple times. Such reflective coatings have been found to produce 95% reflectivity of UV radiation. It will be appreciated that the UV source 140 may instead comprise a single or an array of UV light emitting semiconductor devices such as LEDs generating UV light. A wavelength of two hundred sixty to two hundred sixty-five nanometers (260-265 nm) has been found to be effective in killing or rendering harmless biological contaminants such as viruses, bacteria, and fungi.

The UV light source in this embodiment is powered by means of a power source which, in this embodiment, comprises a battery pack 142. The power source 142 may include a DC to AC converter to facilitate the provision of 120 volts AC or more for powering a mercury vapor lamp from a battery such as a 10 volt DC battery. It will be appreciated that the power supply will include appropriate ballasting circuitry. In the case of LEDs being used as the UV source, the power source will provide the appropriate LED current by means of an appropriate DC voltage converter or through the use of optimized circuitry for LEDs as produced by MAXIM. The battery pack constituting the power supply 142 in this embodiment is packaged integrally with the chamber and includes a charger for the battery pack. However, it will be appreciated that the battery pack could also be separately housed and carried, for example, on a user's belt. It will be appreciated that not only the kill chamber with its sensors and battery pack could be carried separately, but any other elements that are not required to be on the mask 100 could also be carried separate from the mask, e.g., in a backpack, shoulder bag, etc. Thus, for example any cell phone, AM/FM radio, walkie-talkie, or visor information receiver or could be housed carried in a backpack with the kill chamber 110.

The present invention seeks to conserve power while ensuring effective destruction of harmful organic material. In order to conserve power, rate of airflow through the chamber 110 is monitored by means of a flow meter 144, which may be a mechanical flapper, pressure sensor across a venturi, an anemometer, or a mass flow meter. The mass flow meter element produced by MKS Instruments essentially comprises a wire loop that is heated by passing current through it and for which changes in current flow are monitored in order to maintain a substantially constant temperature wire loop. Thus, faster airflow, which will cause greater cooling will require greater current to maintain the temperature of the loop, thereby providing a simple way of measuring air flow rate. It will be appreciated that ambient temperature changes will affect the reading of the mass flow meter. The present embodiment therefore makes use of a second mass flow meter 145 that is exposed to the same ambient temperature but placed in a housing to avoid exposure to air flow, thereby acting as a control device. The differences in reading between the two flow meters will therefore represent a flow rate change. A controller in the form of a microprocessor 146 is connected to the sensor or flow meter 144 to monitor air turnover in the chamber 110 and adjust the UV dosage. The amount of UV radiation to which the air in the chamber 110 is exposed is adjusted by adjusting the radiation source. In one embodiment, a bank or matrix of UV LEDs was switched on and off according to a duty cycle as defined by the microprocessor 146. In addition, in another embodiment, the microprocessor 146 controlled the intensity of some or all of the LEDs in a bank or array of LEDs. In yet another embodiment, the microprocessor 146 selected the number of LEDs that needed to be switch on in order to account for changes in flow rate. It will be appreciated that a combination of two or more such power changes to the LEDs can be implemented.

FIG. 1 also shows a filter 150 provided at the air intake 152 to the chamber 110. The filter 150 reduces the absolute number of living microbes or pathogens that enter the chamber 110 and may have wicking properties or absorbing properties to help reduce the amount of water vapor in the air entering the kill chamber, thereby allowing the UV source to more effectively kill the pathogens in the air

The filter 150 also reduces microbes, dust, or mold entering the chamber 110, thereby reducing contaminants from settling on the chamber's reflective inner surface and compromising its reflective qualities. Since UV light can increase the production of ozone (O₃) and carbon monoxide (CO), the present invention seeks to both monitor and limit the levels of ozone and carbon monoxide. Ozone production can be limited by optically filtering out one hundred eighty-five nanometer (185 nm) UV. Philips, for example, produces a mercury vapor lamp that provides such filtering by providing a titanium-doped glass (type 219 or 230) The carbon monoxide level can be reduced by providing a titanium dioxide layer for chemically reacting with carbon monoxide to produce carbon dioxide (CO₂). In order to avoid the carbon monoxide catalyst material from interfering with the reflective coating material in the chamber 110 the carbon monoxide catalyst is preferably provided in a separate section such as the delivery tube 120 or a portion of the chamber 110 near the outlet 154.

Yet another portion of the chamber 110 may be coated with a catalyst layer such as titanium dioxide (TiO₂) which promotes the breakdown of carbon compounds in the presence of UV light, thereby enhancing the kill effectiveness of the apparatus.

The present invention further includes sensors 160, 162 for monitoring ozone levels and carbon monoxide levels, respectively, in the chamber 110. The signals from the sensors 160, 162 may be sent to a visual display. Preferably, an auditory alarm is included for notifying the user if carbon monoxide or ozone levels exceed a predefined level. In one embodiment, a battery-charge monitor was also included to monitor the amount of battery charge left in the battery pack of power supply 142 and to notify the user both visually and by means of an audible alarm if power levels drop below a predefined minimum charge. As discussed above, this embodiment also includes a UV radiation sensor 172 to detect UV generation failure. The sensor 172 and possibly additional UV sensors also serve to monitor UV radiation and allow adjustment to meet an adequate dose without generating excessive undesirable byproducts. Since the effectiveness of the radiation source is effected by humidity conditions, the present embodiment includes a humidity sensor 192 connected to the controller 146 for controlling the amount of UV radiation pursuant to humidity changes.

As shown in FIG. 1, the mask 130 also includes a microphone 180 to facilitate communication. In order to allow the user to readily use a cellular phone, a cell phone speaker 182 is included in the mask 130 and is either connected to or connectable to a cell phone speaker/ear piece 184. In this embodiment, the mask 100 also includes a walkie-talkie microphone 186 connected to a walkie-talkie speaker 188 to facilitate communication with other workers. The kill chamber 110 also includes an AM/FM radio to allow the user to listen to public announcements and entertainment channels.

Another embodiment of the invention is shown in FIG. 2 in which the chamber 200, instead of having a linear passageway has a wavy passageway to promote turbulent airflow between the inlet 202 and the outlet 204. This ensures that air exposure to the surfaces of the chamber are increased, thereby increasing the effectiveness of the carbon monoxide catalyst. It will be appreciated that the wavy chamber configuration is particularly important in the section of the chamber that is provided with the carbon monoxide catalyst and may, in fact, be limited to this section only.

FIG. 3 shows yet another embodiment of the invention in which baffles 300 are provided in the kill chamber 302 thereby again providing turbulent airflow between the inlet 304 and the outlet 306.

Yet another embodiment is shown in FIG. 4. Here the chamber is divided into narrow passageways 400, each with ultraviolet LEDs 410, thereby ensuring more uniform exposure of the air in the chamber to ultraviolet radiation.

An alternative configuration for the mask and kill chamber is shown in FIG. 5, in which the mask 500 is connected by a flexible delivery tube 502 to a helmet-mounted kill chamber mounted on or integrally formed. In this embodiment, the kill chamber is integrally formed into a helmet 504. It will be appreciated that in another embodiment the chamber could-merely be attached to an outer surface of a helmet such as a bicycle helmet.

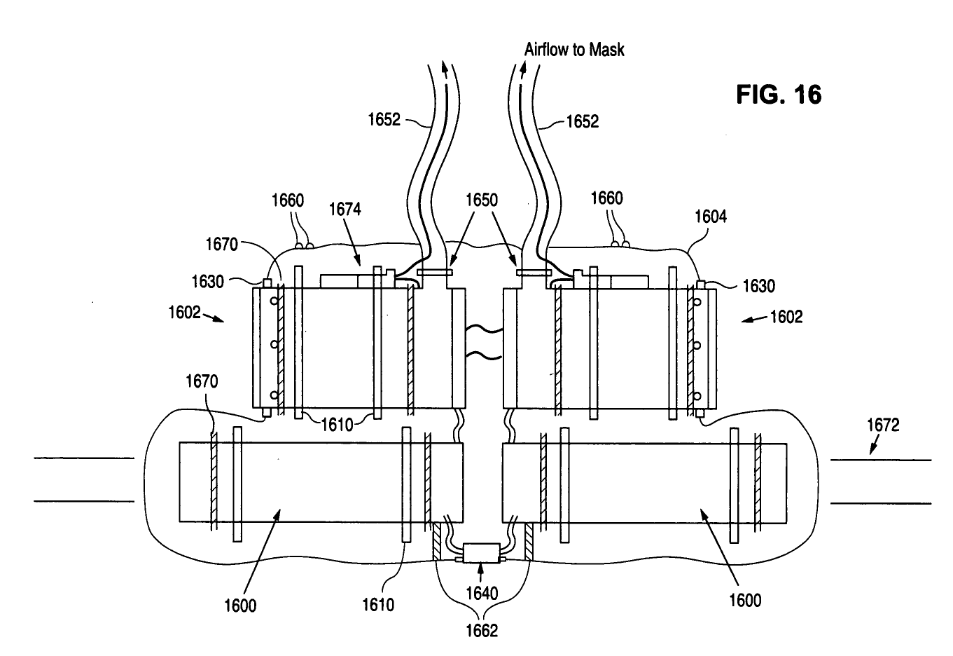

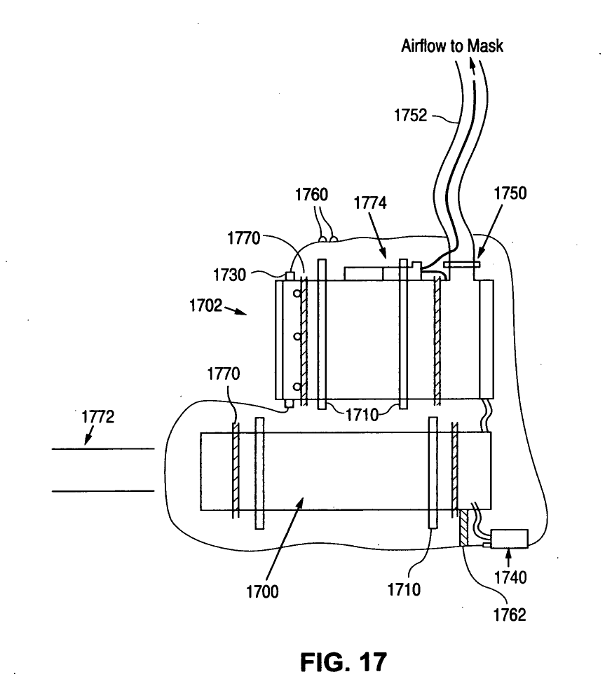

It will be appreciated that the battery pack, instead of being packaged into the helmet 504, may be attached to the user's belt, or to the user's chest, or slung like a purse over the user's shoulder, or carried like a backpack on the user's back, or carried on the user's hips in a hip pouch (fanny pack) arrangement as discussed further below with respect to FIG. 16-17.

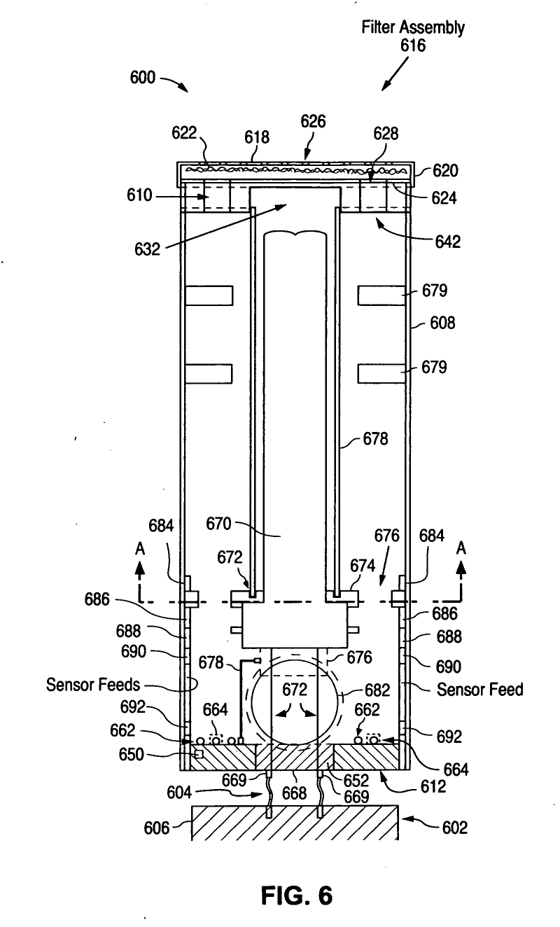

Part of yet another embodiment of the invention is shown in FIG. 6, which includes a UV housing or kill chamber 600 connected to a power supply 602 by means of flexible electrical connectors 604. In this embodiment the power supply 602 comprises several batteries (not shown) housed in a housing 606. Eight Lithium Ion batteries from Sanyo, producing 14.4 V and 64 W Hrs, and weighing 400 grams were used in this embodiment. In another embodiment twelve Nickel Metal Hydride (NiMH) batteries such as the HR-4/3 FAU 4500 were used in this embodiment. These produced 4500 mA hours each for a total of 54 W hours. Thus for approximately a 5W lamp, a 1 Watt air flow pump and some 0.2 W for supporting electronics, the power supply of this embodiment would provide about 8 hours of operation before requiring that the batteries be recharged. These batteries have an 18 mm diameter and are about 67.5 mm long, thus allowing them, in one embodiment, to be packaged into a housing 606 that is about 135 mm×36 mm×54 mm by placing three rows of two batteries on top of a second set of three rows of two batteries. It will be appreciated that other configurations and other types of batteries e.g., NiCd kRF 7000F batteries from Sanyo, or HR-4/3FAU4500 batteries could be used as the power supply 602. In yet another embodiment, a plurality of 13-14 8.9 WHr, UR18650F cells made by Sanyo for providing a total of 115.7-124.6 WHrs were used to power a 9W mercury vapor lamp. It will, however be appreciated that other power sources, including power generators and rechargers could be used e.g., a 110-120 V or 220-240 V wall socket, a DC automobile socket, an AC or DC connection to an electrical supply on boats, ships, aircraft, or trains, a photovoltaic cell or array of cells, or a generator powered by wind, natural gas, propane, gasoline, diesel fuel, alcohol, steam or by man or animal power, e.g., making use of a bicycle or the peddling mechanism of a bicycle.

In the FIG. 6 embodiment the housing of the kill chamber 600 is made of a 4.125 inch long, 2.5 inch inside diameter aluminum pipe or cylinder 608 with a wall thickness of 3 mm and, which is preferably polished on its inner surface to provide a highly UV reflective inner surface. The pipe 608 can even be exposed to a chemical vapor deposition (CVD) process to increase the reflectivity to about 95 percent for UV. Apart from its structural integrity, the aluminum also provides a good thermal conductor for heat generated by the UV lamp 670 and the electronics, which are discussed further below. The pipe 608 defines a housing by being provided with end plugs 610, 612. The end plug 610 is fitted into the upper end of the pipe 608. The pipe also receives a removable filter assembly 616, which together with the end plug 610 will also be referred to as the end cap. The pipe 608 is provided with an outer thread on its upper, outer surface for complementarily engaging the filter assembly 616. The filter assembly comprises a filter housing defined by a filter cap 618 having side walls 620 with an inner thread that engages the outer thread on the pipe 608. The filter cap 618 houses a filter 622 and is closed off by a base plate 624. The filter cap 618 has numerous small holes or air flow passages 626, while the base plate 624 is provided with six 0.65 inch diameter holes 628. In this embodiment the filter 622 is a 0.3 micron particle filter that is equivalent to the N95 3M NIOSH standard.

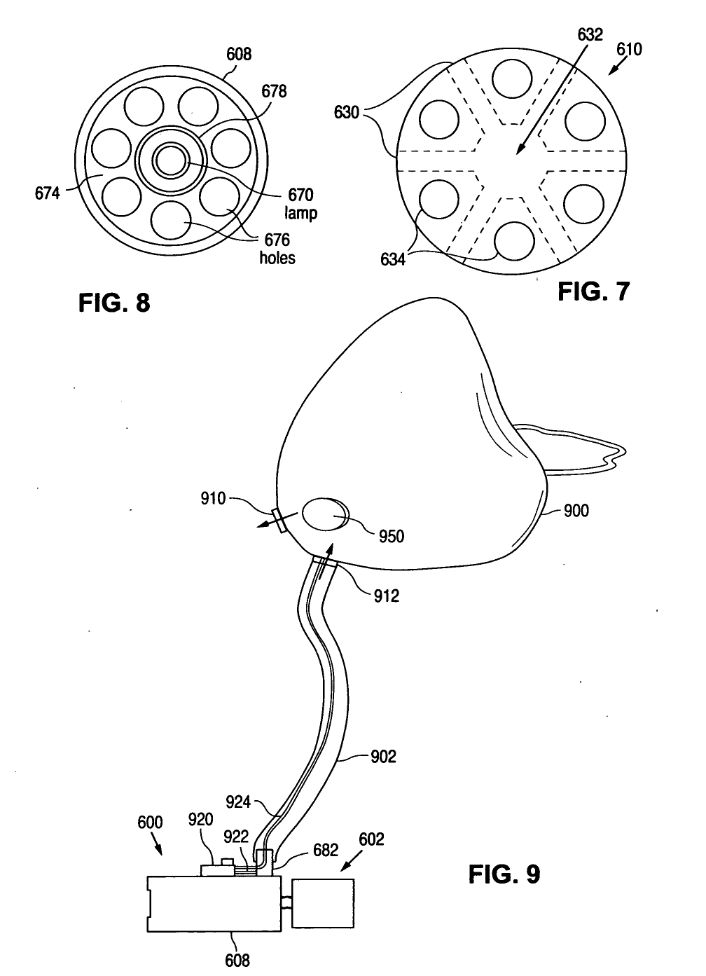

The upper end plug 610 is best understood with respect to the top view of the kill chamber shown in FIG. 7. The plug 610 is made of UV resistant material (e.g., Xylex materials X8210 or X7110 made by General Electric). It defines radial flow passages 630 joining at a central hole 632. The hole 632 and passages 630 allow mercury vapor from the mercury vapor lamp 670 to be vented out of the kill chamber in case the lamp 670 breaks. As discussed further below and as shown in FIG. 6, the kill chamber includes a UV light source in the form of a mercury vapor lamp 670 and a fused quartz sleeve 678 that protects the lamp 670. The sleeve 678 is held in position by the upper plug 610 by fitting into the central hole 632, which, as shown in FIG. 6 extends only partially through the plug 610. The plug 610 is, however, provided with six 0.65 inch diameter holes 642, which pass all the way through the plug and align with holes 628 in the base plate 624 for air flow into the kill chamber surrounding the sleeve 678. The bottom end plug 612 supports the electronics of the air sterilization apparatus, which include a controller or processor, a voltage regulator, and sensor electronics, collectively indicated by reference numeral 650. In this embodiment the plug 612 includes a printed circuit board (PCB) on which the electronics are mounted. The PCB also supports photodetectors 662 for detecting the presence of light with a wavelength greater than 400 nm (visible light), thereby indicating that outside light is penetrating the kill chamber and that the chamber is open to UV radiation leakage. Audible alarms 664 are also provided to produce auditory feedback on various sensor conditions, as is discussed in greater detail below. An electrical connector plug 668 with pins 669 is mounted into the end plug 612 for connecting the flexible electrical connectors 604. As shown in FIG. 6, a UV lamp 670 mounted substantially along the longitudinal axis of the pipe 608 is electrically connected to the pins 669 of the connector plug 668 by means of electrical leads 672.

The lamp 670 should provide about 8 W output and a 253.7 nm wavelength. In this case a G23-2 Pin lamp (PL-S5W/TUV) from Philips, which is a 5W lamp with a 1W output, is mounted on a UV resistant plastic plate 674. The lamp 670 is provided with a ballast 676. Wires 678 extend from a power controller to the ballast 676. The plate 674, which is cemented into the pipe 608 includes a plurality of holes 676 to provide air flow passages as shown more clearly in the sectional view FIG. 8 through the apparatus along line A-A of FIG. 6. The lamp 670 is protected by a UV transparent tube, which in this case is a fused quartz sleeve 678, which surrounds the lamp 670 and is secured between between the plug 610 and an annular groove 672 in the plastic plate 674 to define a lamp housing separate from the air flow region surrounding the sleeve 678. The sleeve 678 of this embodiment has a 3 mm wall thickness, and in this embodiment both the lamp 670 and the sleeve 678 are made of 219 or 230 type, thus including titianium to eliminate the 185 nm line, which produces ozone. Other embodiments simply make use of the 219 or 230 type in either the mercury vapor lamp or the quartz sleeve. In order to ensure that there are no spots or regions in the mercury vapor lamp or quartz sleeve that remain untreated with titanium, the invention proposes that one or both of the lamp and quartz sleeve be supplemented with titanium. This may be done by adding a layer of titanium on the vapor lamp or quartz sleeve or both, by CVD or by implanting titanium e.g. by ion implantation into the vapor lamp or quartz sleeve or both.

As shown in FIG. 6, the kill chamber 600 also includes baffles 679 that are provided as annular plastic disks to promote turbulent air flowing for air flowing through the kill chamber. The annular plastic disks 679 are 0.7 inches thick and have a 1.75 inch diameter central hole.

In order to connect the kill chamber 600 with a face mask (discussed further with respect to FIG. 9) the aluminum tube 608 has a hole in its wall with an outwardly extending flange 682 acting as a hose connector for connecting the hose that leads to the face mask. During use, air is drawn into the housing or chamber 600 surrounding the quartz sleeve 678 through the openings or holes 626 in the filter cap 618, through the filter 622, through the channels 628 in the base plate 624, and through the holes 642 in the top plug 610. The air is then irradiated by UV light from the lamp 670, and passes out of the chamber through the hose connector 682 to the user's face mask. The hose connector 682 includes a quick disconnect for readily disconnecting the hose from the chamber by depressing two tabs or buttons (not shown) on opposite side of the connector for releasing the hose.

As shown in FIG. 6, various sensors are mounted on the inner wall of the tube 608. These include UV sensors in the form of photo detectors 684 for sensing whether and how much UV is being put out by the lamp 670; thermistors 686 acting as hot wire air flow sensors; ozone sensors 688; CO sensors 690; accelerometers 692 for measuring any unwanted jarring or dropping of the apparatus which may have damaged the apparatus. The various sensors are electrically connected to the controller or processor 650 for monitoring the conditions and providing auditory feedback by means of the audible alarms 664 if predefined conditions are not met. For instance, a look-up table can pre-define suitable operating ranges and the controller or processor, which can be a microcontroller, can monitor the sensors and compare the sensor signals to the predefined values or ranges for signaling an alarm if the predefined values or ranges are not met. The audible alarms 664 can, for example, include an audible output generator such as a beeper or voice generator. In addition to the audible alarms, visual alarms, e.g., in the form of LEDs may be attached to an outer container or housing in which the kill chamber is carried.

A block diagram of one embodiment of the electronics is shown in FIG. 12, including the microcontroller, the sensors and the power supply control circuitry for the microcontroller, an air pump (as is discussed in greater detail with respect to FIG. 8) and a mercury vapor lamp as used in the FIG. 6 embodiment. As shown in FIG. 12, this embodiment includes a battery monitor and charge controller chip to avoid overcharging of the batteries in the battery pack that serve as the power supply for the apparatus.

FIG. 9 shows the apparatus 600 with its power supply 602 connected to a mask 900 by means of a flexible pipe or tube 902, which in this embodiment is a ⅝ inch diameter plastic pipe. However, tube diameter and length are preferably adjusted to accommodate size differences and breath volume and breath frequency differences found in children, women and men. The mask 900 covers only the user's nose and mouth and includes a flapper valve 910 for allowing exhaled air to be vented to the atmosphere. The connector or flange on the mask 900 for connecting the hose, or tube 902 includes a quick disconnect requiring two tabs or buttons (not shown) to be depressed on opposite side of the connector for releasing the hose 902. The connector also includes a flapper valve 912 that is spring loaded to bias the valve 912 to a closed position so that the valve 912 is closed when the user exhales to ensure that carbon dioxide rich air is vented to the atmosphere rather than back into the apparatus 600. As shown in FIG. 8, the apparatus 600 also includes an air pump 920, which in this case is a microdiaphragm air pump providing 4-6 standard liters per minute (SLM) at one atmosphere pressure (14.7 pounds per square inch). The pump 920 has an input tube 922 entering the hose connector or flange 682 of the aluminum tube 608 through a hole in the wall of the flange 682. The output from the pump 920 is connected by means of a pipe 924 to the mask 900 by passing through the wall of the flange or hose connector 682 and extending along the inner surface of the tube 902, through the valve 912, and into the face mask 900. In another embodiment, to avoid interfering with the valve 912, the hose 924 may pass out of the pipe 902 near the top of the tube 902 and into the mask In this embodiment the pump 920 is a low pressure pump (e.g. 0.5 to 1.0 inches of water) serving merely to assist the user in breathing and being insufficient to open the valve 912 to the mask 900. In another embodiment the pump 920 is a higher pressure pump (e.g., 1 to 2 inches of water) that not only assists in the breathing process but also forces the valve 914 open and creates a positive pressure in the mask 900 to provide an extra precaution against the ingress of untreated air into the mask 900 along the mask periphery. The higher flow pump also ensures consistent flow rate through the kill chamber. It will be appreciated that by appropriately choosing the tube 902 diameter and length the diaphragm pump 820 can optionally be eliminated altogether, thereby limiting cost and power consumption. It will also be appreciated that by connecting the pump as shown in FIG. 9, natural air flow is not restricted should the pump ever fail.

The invention also proposes including a port or connector to the kill chamber, mask or connecting hose or tube for introducing external substances, e.g. inhalants, nebulizers or atomized medicinal substances. One type of connector would be a pump canister receptor as is commonly known for pump action dispensers. A pump canister connector 950 is, for instance, shown in FIG. 9.

In order to provide an apparatus usable in rural areas or areas where power supplies or charging facilities are not readily available, one embodiment includes a manually operated power source e.g. a hand cranked generator that either charges a set of batteries or directly powers the UV source and other electronics. Such hand cranked generators are currently being used in devices such as portable radios and flashlights.

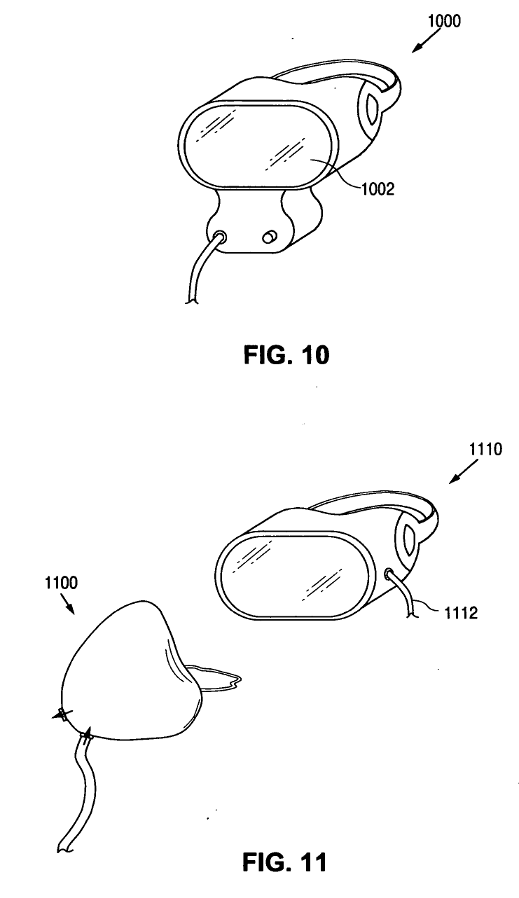

In the FIG. 9 embodiment, in which the mask 900 covers only the nose and mouth, the user will typically wear safety glasses or goggles to protect the eyes. In another embodiment, shown in FIG. 10, the mask assembly 1000 includes goggles 1002 having scratch resistant plastic lenses and provided with a UV coating, thereby allowing the user to enter a UV decontamination chamber without fear of damaging his or her eyes. In order to avoid subsequent contamination of the user after use of the apparatus, some or all of the face mask and goggles may be made to be disposable or may be made from a UV resistant material or covered by a UV resistant coating to permit decontamination of the face mask and goggles in a UV decontamination chamber. Instead, if the face mask and goggles are intended for decontamination by means of a chemical agent such as bleach (e.g., Clorox), the mask and goggles may be made of a material resistant to such chemical agent.

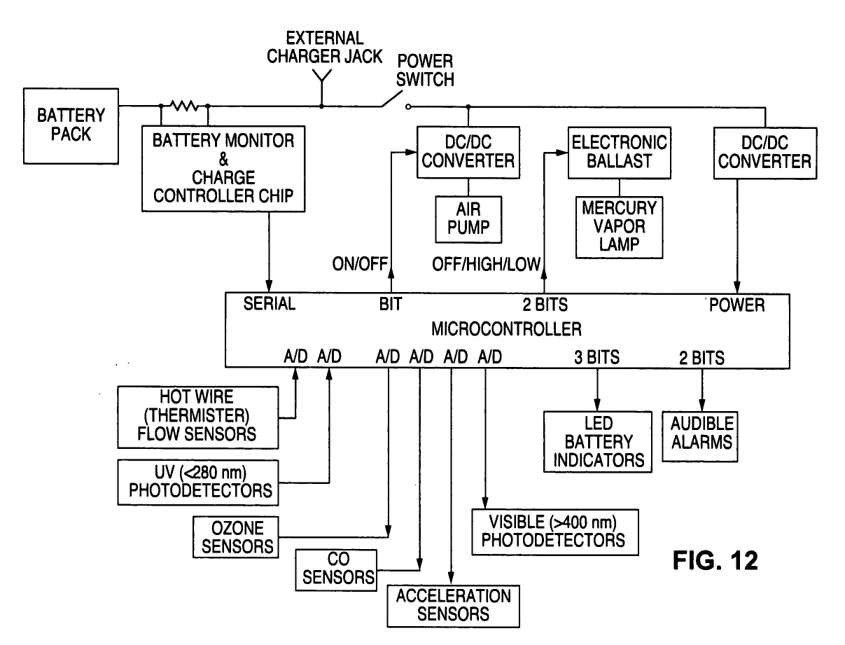

In yet another embodiment, shown in FIG. 11 the mask 1100 again covers only the mouth and nose, but in this embodiment the system includes goggles 1110 that seal to the user's face and are provided with a separate air tube 1112 that is fed by an air pump such as the diaphragm pump 920 or by a separate air pump that pumps UV treated air.

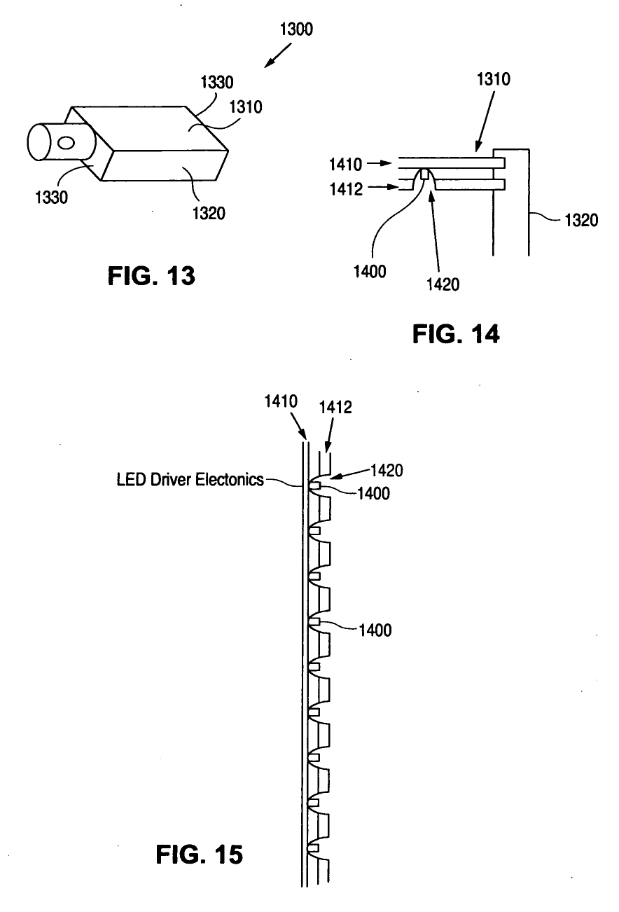

While the embodiment of FIG. 6 makes use of a mercury vapor lamp 670, the UV source could also be provided by LEDs as shown in FIGS. 13-15. For instance, InAlGaN LEDs with a dominant wavelength of 265 nm were used in one embodiment. Sources vary somewhat in calculating the exact amount of radiation required in order to kill avian flu, for instance. One approach is based on energy per unit area required to destroy DNA or RNA. Using this approach, 6600 uWsec/cm² is required to achieve the desired destruction of DNA or RNA. Typically an adult at rest will take 10 breaths/minute, thus providing a 6 second chamber time for each breath (assuming flow is not controlled by an air flow pump). A tubular light source providing 1.0 watt of UV radiation located concentrically in an 8 cm diameter tube that is 16 cm long will provide a radiation intensity at the cylindrical wall surface of 1/804=1243 uW/cm² since the wall has a surface area of 804 cm². This intensity over six seconds will provide a radiation dosage at the wall of 7458 uWsec/cm² for each breath. Thus a 1.0 W output source will provide a safety factor for kill of about 7460/6600=1.13. This calculation underestimates the average dosage level. The average dosage will be higher due to greater radiation intensities closer to the bulb and reflection of radiation from the wall. For example, if 95% of the radiation is reflected from the wall, then average dosage at the wall will be at least 7458*1.95 or 14543 uWsec/cm² or a safety factor of at least 2.2. An LED will produce about 1 mW output. Thus the source would need about 1000 LEDs to provide the 1.0 W output considered above if the LEDs are arranged in a cylindrical array. If a safety factor of approximately 1.0 were considered sufficient, or if linear arrays were used such that the LEDs illuminate a long, reflective light path then many fewer LEDs would suffice. At 40 mA per LED, 1000 LEDs would draw 40 A of current and at 6V would require approximately 240 W of power. It will, also, be appreciated that the need for an accelerometer to monitor shocks that might damage a non-LED UV light source such as a mercury vapor lamp will be less critical when UV LEDs are used. In this embodiment the bank of LEDs 1400 is mounted along one or more of the inner walls of a rectangular housing 1300 as shown in FIG. 13. The two main sides 1310 of the rectangular housing 1300 are made of double sheets of aluminum 1410 shown in FIGS. 14 and 15, which fit into slots in UV resistant plastic panels 1320 forming the narrow sides of the housing. The ends 1330 of the housing are also made of UV resistant plastic and also receive the aluminum plates 1410 in slots. As shown in FIGS. 14 and 15, the UV LEDs 1400 are mounted in cups 1420 between the outer aluminum plate 1410 and the inner aluminum plate 1412, which is polished, is provided with holes to expose the cups 1420.

As discussed above, the kill chamber and power supply can be carried in a fanny pack or hip pouch. Two embodiments of such a fanny pack arrangement are shown in FIG. 16-17. FIG. 16 shows dual battery packs 1600 constituting the power supply, and dual kill chambers 1602 housed in a fanny pack 1604. The dual nature of the kill chambers and battery packs provides for redundancy in case of failure of one or other of the components. In addition, both the battery packs 1600 and kill chambers 1602 are protected by annular foam disks 1610. The foam discs 1610 also serve to space the battery packs 1600 and kill chambers 1602 from the wall of the fanny pack for better air flow past the battery packs and kill chambers. The battery packs and kill chambers are secured relative to the fanny pack walls by means of elastic bands 1670 attached to the inner walls of the fanny pack. The kill chambers 1602 have their air inputs in flow communication with the outside by providing holes on opposite sides of the fanny pack 1604 and mounting the chambers 1602 by means of brackets 1630. In this embodiment, the battery packs 1600 are cooled by cooling exhaust feed lines from two micromotors with fans 1640, which are mounted between two plastic supports 1662 that are 2 inches in diameter. The embodiment of FIG. 16 also includes shut-off valves 1650 controlled by the controller of the kill chamber electronics. The valves 1650 close the tubes 1652 leading to the mask (not shown) in the event that a mercury vapor lamp breaks, i.e., if the UV detectors detect no UV radiation from a particular UV lamp. Also, if failure or inadequate UV radiation is detected, e.g., if the UV photodetectors detect no UV radiation within one or both of the kill chambers or the radiation level falls below a predefined level, alarms can be activated. As mentioned above, alarm conditions can be both visually and audibly identified. In this embodiment, LEDs 1660 are mounted on the fanny pack to provide the user with visual alarm information. Here several LEDs are provided, with different colors to provide different types of information. For instance, a green, amber and red LED can be provided for the kill chamber to indicate that the unit is good to use, or that the pump is down, or that no UV is being generated, respectively. Similarly, a green, amber and red LED can be provided for the battery pack to indicate, for example, that the battery pack is adequately charged (more than ½ hour left), or has less than ½ hour left, or has less than 5 minutes left, respectively. Regarding the shut-off valve, it will be appreciated that the shut-off valve 1650 is not necessary where the UV light source is provided by a bank of UV LEDs. Also, by providing the connection between the tube 1652 and the face mask as a separable connection and providing the connection with a quick release connector, e.g. oppositely positioned depressable tabs on the connector, the tube can readily be removed in the event that the shut-off valve 1650 is closed. In one embodiment the fanny pack includes a pouch or Velcro patch for holding a snap-on particle filter that is attachable to the face mask using a similar quick-release connector, once the tube 1652 is removed. One feature of the invention is to provide quick release connectors such as the depressable tab or depressable button connectors described above or other connectors that allow the kill chamber, delivery tube, face mask, and filter to be readily separated, thereby defining a modular apparatus that allows parts to be exchanged or replaced. In the FIG. 16 embodiment the fanny pack 1604 is made from waterproof material on its top and sides for helping to contain any mercury vapor in the event of a mercury vapor lamp breakage. The fanny pack 1604, however, is provided with a mesh bottom portion to help with air circulation and cooling of the battery packs 1600 and kill chambers 1602 and to provide visible light to the kill chamber, thereby allowing the photodetector 662 (FIG. 6) to monitor visible light entering the chamber, which would indicate a leak constituting a UV radiation hazard.

Another embodiment of the fanny pack arrangement is shown in FIG. 17. In this embodiment there is no redundancy shown, but the power supplies 1700 and kill chambers 1702 are again mounted in parallel. The battery pack 1700 and kill chamber 1702 are protected by annular foam disks 1710. The foam discs 1710 also serve to space the battery pack 1700 and kill chamber 1702 from the wall of the fanny pack for better air flow past the battery packs and kill chambers. The battery packs and kill chambers are secured relative to the fanny pack walls by means of elastic bands 1770 attached to the inner walls of the fanny pack. The kill chambers 1702 have their air inputs in flow communication with the outside by providing holes on opposite sides of the fanny pack and mounting the chambers 1702 by means of brackets 1730. In this embodiment, the battery pack 1700 is cooled by cooling exhaust feed lines from micromotor with fan 1740, which is mounted next to a plastic support 1762 that is 2 inches in diameter and acts as a spacer between the power supply and the fanny pack. The embodiment of FIG. 17 also includes shut-off valves 1750 controlled by the controller of the kill chamber electronics. The valves 1750 close the tubes 1752 leading to the mask (not shown) in the event that a mercury vapor lamp breaks, i.e., if the UV detectors detect no UV radiation from a particular UV lamp. Also, if failure or inadequate UV radiation is detected, e.g., if the UV photodetectors detect no UV radiation within one or both of the kill chambers or the radiation level falls below a predefined level, alarms can be activated. As mentioned above, alarm conditions can be both visually and audibly identified. In one embodiment, the battery backpack 1700 was made detachable from the rest of the apparatus to permit replacement with a new battery pack. In another embodiment, the batteries in the backpack were arranged for easy access and sliding contacts mounted in the backpack for easy removal and replacement of the batteries in the backpack of the battery backpack 1700. In addition, in one embodiment, an electrical access cable to the batteries was provided for charging of the batteries at any suitable electrical outlet when the user was not moving around.

While the flexible connector hose 902 of FIG. 9 was a circular cross-section hose, other embodiments are also proposed by the present invention. For instance, a low profile rectangular, non-collapsible, yet flexible airline could be used instead. This could be worn beneath clothing and connect to the side of the mask to reduce snagging.

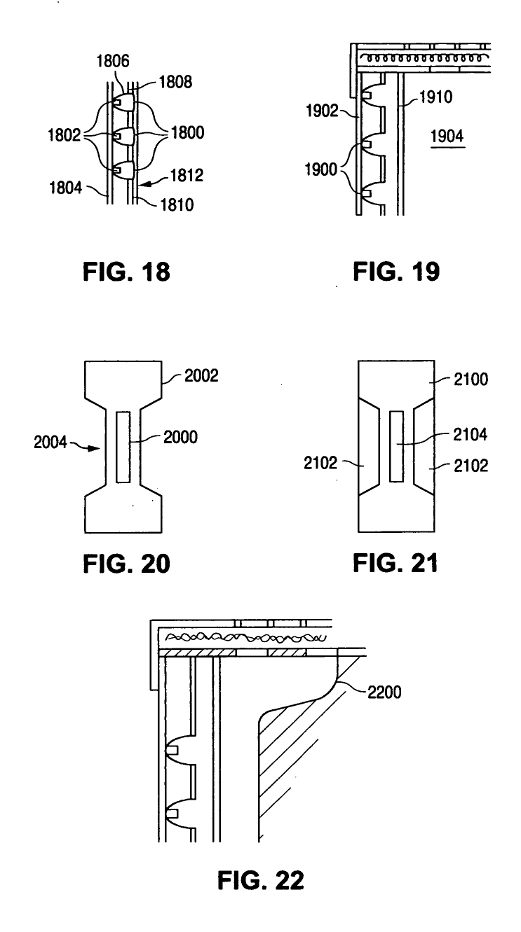

In order to protect UV LEDs against back reflection of UV light, one embodiment of the invention, shown in FIG. 18 includes polarized glass lenses 1800 covering the LEDs 1802 that are attached to the kill chamber housing wall 1804. As in the FIG. 14 embodiment, the LEDs 1802 are housed in cups 1806 mounted in holes in an inner aluminum wall 1808. In this embodiment, in addition to a highly reflective coating 1810 (which will be discussed in greater detail below) the inner aluminum wall is provided with a coating 1812 that is transparent to UV hand has a high refractive index. This has the effect of bending the incident UV light away from the normal to the surface and thus increases the reflective effect by reflecting the light at a greater angle than the angle at which it came in.

Another embodiment for protecting the LEDs against UV light is shown in FIG. 19 in which the LEDs 1900 mounted on the aluminum housing wall 1902 are separated from the air flow chamber 1904 by a polarized, UV transparent tube 1910 (in this case a glass tube).

Since light power density diminishes by the square of the distance from a point source of light and by 1/d for an array form of light emitter such as a linear cylindrical tube, it is desirable for maximum killing capacity, to have air flow within the kill chamber pass as close to the UV light source as possible to ensure that biological contaminants are exposed to sufficient light power. One embodiment for a housing for achieving this purpose in the case of a kill chamber with a central mercury vapor lamp is shown in FIG. 20, which shows the housing in non-cylindrical form. Along the length of the mercury vapor lamp 2000 the housing 2002 has a narrowed or constricted portion 2004 to ensure air flow close to the mercury vapor lamp 2000.

In another embodiment, shown in FIG. 21, a cylindrical housing 2100 is adapted to provide the constriction by including an annular baffle 2102 that surrounds the UV light bulb 2104.

In the case of a kill chamber that makes use of UV LEDs as the light source, such as the embodiment shown in FIG. 19, air flow close to the light source is ensured by including a central baffle 2200 as shown in FIG. 22.

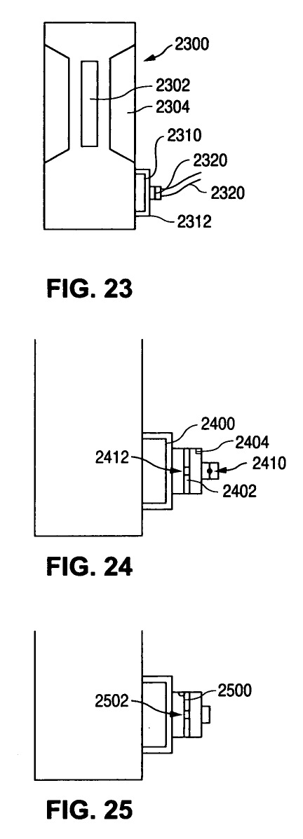

While the above embodiments of the kill chamber of the invention have discussed the use of a pump to provide or supplement air flow to the face mask, air flow may instead be provided by making use of a fan or blower as shown in FIG. 23. In one embodiment a centrifugal blower was used, in particular a GB1205PKV1-8AY by Sunon (www.sunon.com) available from Jameco. In FIG. 23 the kill chamber is shown in simplified form and depicted generally by reference numeral 2300. While the mercury light bulb 2302 and baffle 2304 are shown the filter cap detail and light bulb connector are left out for simplicity. In this embodiment a fan or blower 2310 is provided in a housing 2312, which is in flow communication with the inside of the kill chamber 2300 and a hose 2320 that leads to the face mask (not shown). In accordance with the definition provided above, the fan may be mounted directly in the housing 2312 or may include its own housing, thereby defining a blower that is mounted in the housing 2312.

The blower or fan 2310 in this embodiment is implemented as a constant flow arrangement in which the flow rate remains substantially constant whether the user inhales or exhales. The constant flow arrangement includes a control valve, which may simply be a mechanically adjustable valve to achieve the desired flow e.g. manual butterfly or gate valve, but in this embodiment is an electrically controllable valve 2320 that is controlled by a signal obtained from a flow transducer (eg., based on a Venturi pipe).

As shown in FIG. 24, the blower or fan 2400 may instead be implemented as a constant pressure arrangement in which the flow rate varies as the user inhales or exhales, but pressure is adjusted to maintain substantially constant pressure. This may be achieved by measuring the pressure behind a one-way valve e.g., by measuring pressure behind a flapper valve 2402 electronically using a solid state pressure transducers 2404 and varying the flow of air provided by the fan or blower by varying current to the fan or blower 2400. This constant pressure arrangement may be used not only to control a fan or blower but may also be used for controlling air flow provided by a pump (which typically uses about 11 W to produce 20 standard liters per minute (SLM)). In contrast a fan or blower uses only about 1-2W to produce 80 (SLM).

Instead of controlling current to the fan or blower or to the pump, the flow rate can be adjusted for constant pressure by varying the position of an electrically-controlled valve or shuttering mechanism, referred to herein as a control valve, such as a butterfly valve, or gate valve etc., that is located in the delivery tube or at the output side of the fan, blower, or pump or at the inlet to the face mask. Such a valve mounted at the output of the kill chamber is shown in FIG. 24 and depicted by reference numeral 2410. In another embodiment the valve was mounted in the delivery tube itself. The valve may include a latch, e.g., a magnetic latch to reduce electric power consumption by the valve. Nevertheless, it will be appreciated that if constant pressure is maintained using only an electrically actuated valve such as the valve 2410, the blower, fan or pump may be wasting energy by blowing or pumping more than required, and may create excessive pressure of the inlet air. Thus control of the blower, fan or pump current is advisable. As part of the modular nature of the preferred embodiment, the pressure sensor is preferable releasably connected. For simplicity, in another embodiment instead of electronically controlling the shuttering mechanism or control valve 2402, the invention may be implemented by using a mechanical shuttering mechanism or control valve 2500 as shown in FIG. 25, e.g., a spring loaded valve that opens up at defined pressure. Preferably a controlled leak is provided into the mask to ensure positive pressure e.g. 0.5 SLM. The controlled leak may be implemented by forming a hole 2412 or 2502 in the valve 2402 or 2500, respectively. One advantage of a constant pressure implementation is that during low flow rates (e.g. during exhaling or lower user activity) any biohazards such as viruses entering the chamber continue to be radiated for longer periods of time. It will be appreciated that in embodiments where the control valve also acts as shut-off valve in the event of UV lamp breakage, the control valve does not have a hole 2412, 2502. Instead, the control valve is simply controlled to remain slightly open at all times except when UV lamp breakage is detected.

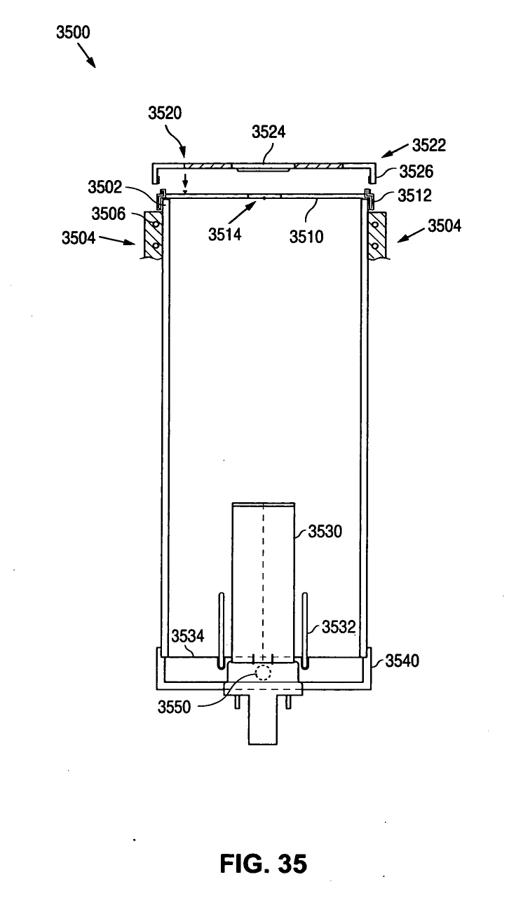

Another embodiment of a kill chamber according to the invention is shown in FIG. 35. The kill chamber 3500 includes an aluminum housing 3501 made of two halves 3502 with outwardly extending flanges 3504. In this embodiment, the aluminum halves are made using 0.1 inch thick aluminum sheets with a UV reflective coating comprising 0.5-1 μm thick sputtered Al and 0.5 μm thick SiO₂ using a rocker during sputtering. The aluminum sheets were pressed into shape using a polished steel tool and die. The flanges, in this embodiment, are provided with internally threaded holes 3506 for receiving complementarily threaded screws. It will be appreciated that the holes could instead have a smooth inner surface, in which case the two halves could be held together by bolts and nuts. In addition, a high temperature vacuum sealant or glue is provided between the opposing flanges to ensure an air-tight seal. The top of the housing is partially closed by a disk 3510, which in this embodiment is formed from a Pyrex dielectric stack with a 250-270 nm wavelength high reflectivity coating. The disk 3510 includes a central hole 3514 defining an air inlet to the chamber 3500. The disk 3510, in this embodiment is held in place by a UV resistant plastic ring 3512 made by General Electric (GE), which extends by about 0.125 inches above the rim of the aluminum chamber housing to define an air space as is discussed in greater detail below. The ring 3512 is provided with outer threads along its upper surface for receiving a filter cartridge discussed in greater detail below. The plastic of the ring 3512 has the advantage that it is UV resistant and does not absorb water.

The filter in this embodiment comprises an annular or doughnut-shaped N100 filter cartridge which includes a plastic cap 3522 made from GE ultraviolet resistant plastic. The cap 3522 supports a central annular filter element 3520. The central portion of the annular filter element 3520 is, in turn, closed off by a central circular plastic disc 3524 having a MgF₂ or other reflective coating 3525 on its lower surface.

The cap 3522 includes side walls 3526 with inner threads allowing it to be screwed onto the top of the chamber housing by engaging complementary threads on the outer surface of the chamber housing 3501. The plastic ring 3512 also has grooves on its outer upper surface for engaging the threads on the side walls 3526 of the cap 3522. Once attached, the cap 3522 defines a gap or space between itself and the disk 3510 due to the ring 3512 which extends above the rim of the aluminum housing. Air passing through the filter 3520 therefore passes through this gap to the air inlet 3514.

The air then passes into the chamber, which in this embodiment has a volume of 0.86 liters and a 380 cm² surface with average reflectivity for UV of 90%. The mercury vapor lamp 3530 in this embodiment is either a 5W or 9W single connector UVC bulb with 185 nm line suppressed. The lamp 3530 is surrounded at its lower end by a 1.25 inch diameter SiO₂ sleeve or tube 3532 that is 1-5 nm thick, has an aluminum coating that is 500-3000 Angstrom thick. The tube 3532, which can also be made of other reflective material e.g., polished aluminum to channel the UV rays, is supported in a groove formed in a UV resistant plastic disk 3535 which supports an aluminum disk 3534. The sleeve 3532 is secured in the groove by means of a high temperature sealant.

The lower end of the aluminum housing 3501 is closed off by means of a plastic cap 3540 made of GE UV resistant plastic with 0% water absorption, and which has a central hole for receiving the lamp 3530. Vertically extending holes 3548 are also formed the plastic cap 3540 and aluminum disk 3534 to channel air from the chamber to horizontally extending holes 3550 formed in the plastic cap 3540 and in the walls of the housing 3501.

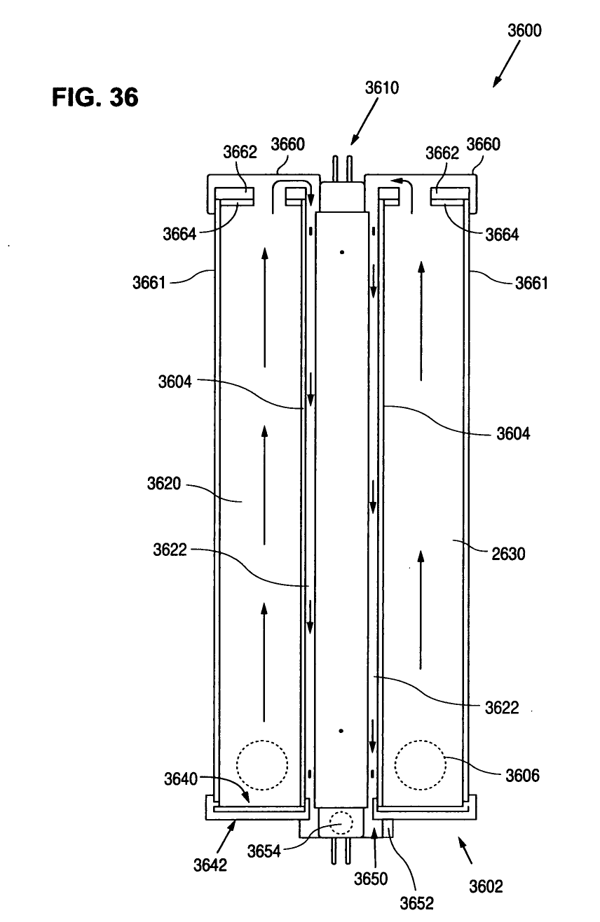

Yet another embodiment of a kill chamber of the invention is shown in FIG. 36, which makes use of a housing 3600 in which both the air inlet and outlet are formed at the same end 3602. As in the previous embodiment, the kill chamber in this embodiment is made in two sections from 0.1 inch pressed aluminum that are formed into cylinder halves using tool and die. For ease of description, the flanges are not shown in this embodiment. The kill chamber in this embodiment, however, is divided into concentric channels by means of a quartz tube 3604 that surrounds the double connector 6W LVC bulb 3610. The air enters the air inlet opening 3606 via a filter cap (not shown) that attaches over the air inlet opening 3606. The air then passes up the outer channel 3620, over the top of the tube 3604 and down the inner channel 3622, allowing it to pass very closely to the UV lamp 3610. As shown in FIG. 36, the outer channel is covered at the lower end of the housing by an annular aluminum disk 3640 which is held in place by an annular plastic cap 3642. This allows air coming down the inner channel 3622 to pass into an outlet chamber 3650 defined by a plastic cap 3652. A hole 3654 in the cap 3652 receives a blower (not shown) for moving the air through the kill chamber and along the delivery tube (not shown) to the face mask (not shown). The upper end of the housing 3600, on the other hand, is closed off by an annular plastic cap 3660 that attaches to the aluminum wall of the housing and is provided with secondary inner wall 3662 for supporting the quartz tube 3604 while maintaining an air channel 3663 between the outer wall of the cap and the inner wall 3662. The inner wall 3662 is provided with a UV reflective aluminum disk 3664 on its lower surface for reflecting UV light from the lamp 3610. In order to allow the air to flow from the outer channel 3620 to the channel 3663, the inner wall 3662 and aluminum disk 3664 are provided with holes 3667. The air then passes from the channel 3663 to the inner channel 3622 through holes 3669 formed in the inner wall 3662.

Yet another embodiment of the invention is shown in FIG. 37. The housing in this embodiment comprises a frusto-conical section 3702 made of polished aluminum and housing a mercury vapor lamp 3710. The housing further includes a cylindrical section 3704 also made of polished aluminum. The lamp 3710 is secured by means of a plastic ring joint 3712 made of UV resistant plastic. A second ring joint 3714 connects the sections 3702, 3704 and supports a SiO₂ lens 3720 which focuses the UV beam from the lamp 3710 toward a high intensity zone 3730. The focused beam is indicated by reference numeral 3732, and the zone comprises a hole or channel 3740 formed in a UV resistant plastic plug 3742. The depth of the hole 3740 is chosen to provide sufficient exposure to the pathogens by the focused UV beam as the air stream with the pathogens flows through the hole 3740 into the chamber 3700. In this embodiment the hole 3740 has a diameter of 1 cm. The top of the chamber is closed off by a filter cap assembly 3750 comprising a filter 3752 held in a plastic cap 3754. The cap 3754 is threaded to engage complementary threads on the outer surface of the plug 3742. In order to reflect the UV light back, a polished aluminum disk 3756 is mounted between the filter assembly 3750 and the plug 3742. Holes are formed along the outer periphery of the aluminum disk 3756 to allow air to pass from the filter into the hole 3740. The air then passes down the section 3704 of the kill chamber and through a hole 3760 in the lens 3720. The air passes down the section 3702 and out of the kill chamber via a hole 3780 formed in the wall of the section 3702. To ensure good reflection, this embodiment also has a polished aluminum disk 3790 attached to the lower surface of the plug 3742. The nature of the filter may be chosen to limit clusters or clumps of the particular biological pathogen(s) that the UV light source is intended to kill or destroy. Typically a filter capable of filtering 0.1 μm diameter or smaller pathogens is used. The purpose of the high intensity zone 3730 is to address biological contaminants with a higher resistance to UV radiation. While in this embodiment the high intensity zone is defined at the input, it could also be defined at the output to the housing or any other location in the housing or upstream or downstream of the housing. As described above, the lens 3720 acts as a UV beam magnifier to focus the beam onto the high intensity zone. The beam magnifier typically includes a lens made of high transmissivity material, in this case silicon dioxide. Instead of a UV mercury vapor lamp, a UV laser or a flash lamp (e.g., xenon or xenon-mercury flash lamp produced by Perkin Elmer such as the RSL3100) producing a high intensity burst of UV light or other energy source may be used. In such a case, the beam magnifier may still be used with the UV laser or flash lamp. In the case of a flash lamp the hole depth is chosen to ensure that no air passes all the way through the hole without being exposed to at least one pulse. The systems described above for supplying air may be portable and may be powered by one or more replaceable or rechargeable batteries, e.g., lithium ion batteries.

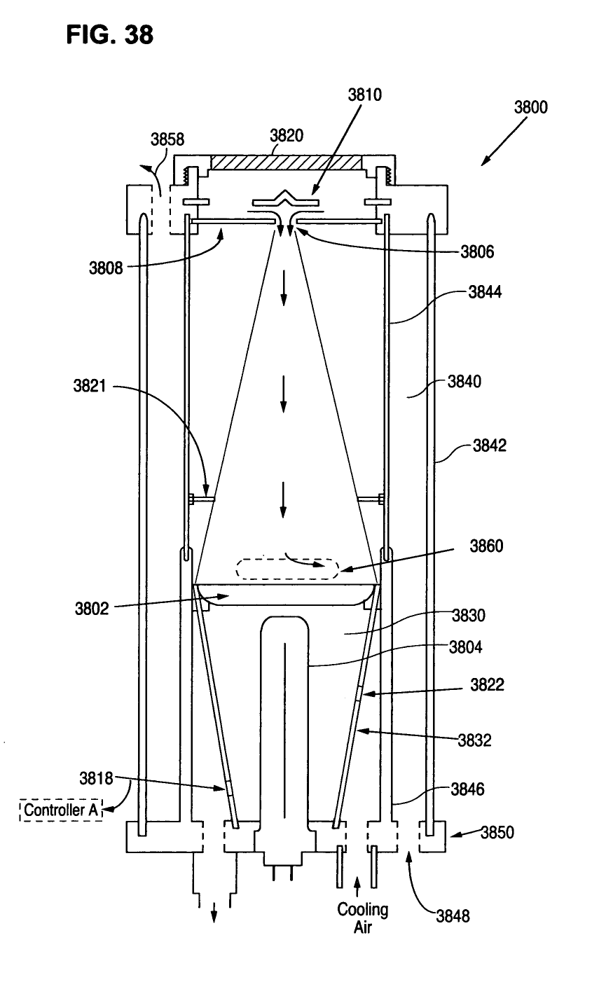

Further embodiments of the invention are shown in FIGS. 38-44. The embodiments of FIGS. 37-44 deal with portable devices for use with a face mask. Thus they each device is intended to provide sterilized air to a specific user. However, the concepts of providing a high intensity zone in which higher intensity radiation is provided to a certain region, in accordance with the invention, is applicable also to other air sterilization systems such as UV sterilizers in air ducts of buildings, airplanes, ships, trains, etc.

The embodiment of FIG. 38 specifically provides for sterilization not only of air from the atmosphere that is to be supplied to a specific user via a face mask (not shown) but also of air exhaled by the user. The kill chamber 3800 includes an SiO₂ lens 3802 that acts as a beam magnifier to focus 0.8 Watts from a 5 Watt (input power) Philips LWC lamp 3804 onto an incoming air stream that flows into the chamber 3800 through a 7 mm diameter hole 3806. The hole 3806 is formed in an SiO² disk 3808 with an aluminum deposition on the side facing the lamp 3804, and defines a first input to the kill chamber. The hole 3806 confines air flow to a narrow input region that coincides with the focal point of the lens 3802 and thus defines a high intensity zone in which pathogens are exposed to high intensity radiation. The 253.7 nm UVC is reflected by a polished aluminum disk 3810 (90% reflectivity) resulting in an intensity of 4.0 million microjoules of 253.7 mn UVC energy per cm² in the incoming air. This high intensity exposure is followed by a dose of 12 479 microjoules (combined direct or front radiation and reflected or back radiation) at a flow rate of 1.5 standard liters per second (90 SLM). It will be appreciated that, in this embodiment, since the UV light from the lamp 3804 is essentially funneled by the lens 3802 and the air stream that passes through the HEPA filter 3820 is funneled by the hole 3806 and a 1.5 inch diameter hole in a second disk 3821, the high intensity radiation is, in fact, a range of radiation intensities that gradually decreases from its highest value at the focal point to its lowest value at the lens surface, before passing out of the housing via holes 3860. Air flow from through the HEPA filter 3820, into the housing of the kill chamber 3800 and out of the hoes 3860 to the face mask is generated by a blower (not shown) connected to the outer surface of the kill chamber housing.

In this embodiment a separate lamp housing 3830 is defined by a frusto-conical aluminum reflector 3832. The lamp housing 3830 is temperature controlled by averaging the closest 2 or 3 temperature sensors 3818 to sweep the temperature range between 35 C and 45 C, and creating a cooling air flow using an in-line cooling fan (not shown) to fix the temperature at the level where the UVC output (measured by averaging the closest 2 of 3 UVC photodetectors 3822) is a maximum. In addition to the air from the atmosphere, which passes the HEPA filter 3820 being sterilized, exhaled air from the user's face mask is pulled through an outer annular region 3840 that is defined between the aluminum housing wall 3842 and an inner sleeve. The housing wall in this embodiment, is made from a 3 inch diameter aluminum tube with 0.1 inch wall thickness and polished inner surface. The inner sleeve is made from a fuse quartz tube 3844 with 2 inch inner diameter and 3 mm wall thickness. The lower portion of the inner sleeve is defined by a GE UV plastic tube that supports the tube 3844. The exhaled air is pulled through the annular region 3840 by a small continuous exhale blower (not shown) through a one-way valve in the user's face mask (not shown) and enters the outer annular region 3840 through an opening 3848 in a lower GE UV plastic end cap 3850, and is expelled from the kill chamber 3800 through an opening 3858 in an upper GE UV plastic end cap 3860. The exhaled air, in this embodiment, is exposed to a dose of more than 8000 microjoules of UV radiation.

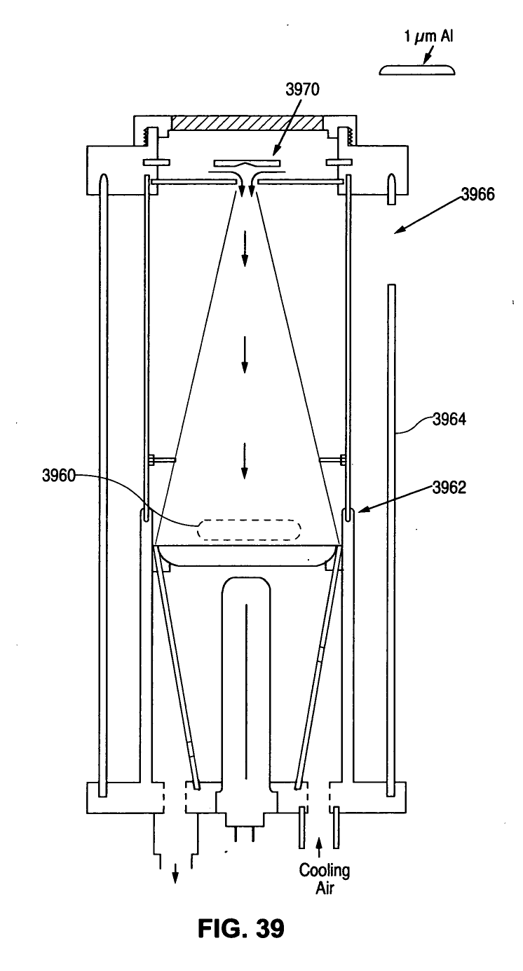

FIG. 39 shows another embodiment of the invention that is similar to the FIG. 38 embodiment However, in this embodiment there is no separate air inlet for exhaled air that is exhaled by the user. Instead, a separate kill chamber would be used to sterilize the exhaled air in this embodiment. Also, in this embodiment the air outlet is defined by 4 holes 3960 formed in the inner sleeve 3962. The air then passes out of the aluminum housing 3964 through a 3 inch diameter hole 3966. Thus, unlike the FIG. 38 embodiment, the air first passes up the outer chamber 3962 before leaving the housing 3964. This allows the air to be exposed to further UV radiation, as discussed in greater detail below. As in the previous embodiment, the chamber includes an SiO² lens having more than 99% transmissivity that is used to focus 0.8 W from a 5 W (input power) Philips UVC lamp on an incoming air stream flowing through a 7 mm diameter hole. In other embodiments hole diameters of 0.5 to 1 cm were used. In contrast to the FIG. 38 embodiment, the 253.7 nm UVC light in this embodiment is reflected by an Edmund Scientific SiO2 lens 3970 with a 1 μm Al deposition on the lower (collection) side. The projection of the UVC beam and its back reflection results in an intensity of 4 million micorojoules/cm² of UVC light being projected on the incoming air. This includes 800,000 microjoules/cm² incident radiation and 92% reflection (or 736,000 microjoules/cm²) for a total of 1,536,000 microjoules/cm². Over 0.7 cm diameter hole, the total exposure of air passing through the hole is 4000,000 Joules. The high intensity radiation is effective in destroying pathogens described in the multi-hit model where clusters form and require a higher than expected number of hits to destroy the pathogens. As the intensity I→∞, the time t→0, thus allowing a relatively short exposure time to a high intensity radiation to destroy the pathogens that would not be destroyed by the dose described in the classical model S=e^{-kIt} where S is the remaining pathogens and k is the constant for a specific pathogen response to UVC light. The high intensity radiation is followed by a dose of 12479 microjoules/cm²at a flow rate of 1.5 standard liters of air per second (90 SLM). This does not include any value for photon transmission back into the quartz chamber 3972 due to reflection off the polished aluminum inner surface of the housing tube 3964. Nevertheless, this dose is well above the 6600 microjoules/cm² required to destroy the influenza A subtypes and yields a remaining pathogen population below 8×10−7 using S=e−kIt for influenza A because of the average 1>80 000/cm². In this embodiment, it receives a further UVC dose in the outer 0.32 liter chamber 3962. The outer chamber 3962 adds effective volume providing much higher doses at more typical respiration rates of 30-40 SLM.

In another embodiment, instead of making use of a beam magnifier, a high intensity zone was created by providing a flash lamp or high power mercury vapor lamp in addition to a low power UV lamp such as the lamp 3804.

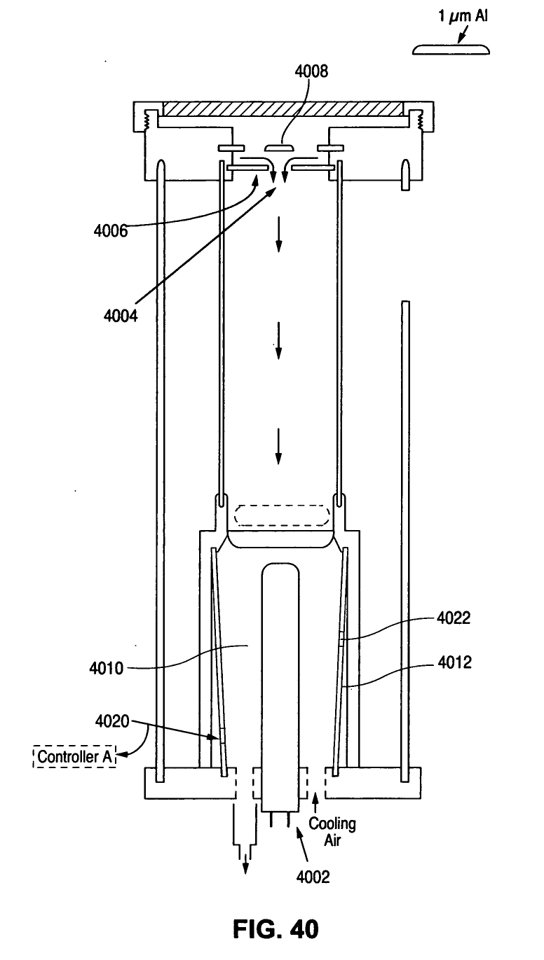

In FIG. 40, like the embodiments of FIGS. 37-39 also shows a design that defines a high intensity zone. It exposes all incoming air to a highly uniform, extremely high intensity flux of UVC light. Kowalski explains that the time t to destroy the secondary survival populations may approach zero at high intensities. This is demonstrated in the survival curves shown for aspergillus niger (a difficult pathogen to kill with UVC light) {Kowalski et al, 2000} where the surviving population drops from nearly 1 to 0.001 as the intensity increases from 200 micro joules per cm sq. to 1800 micro joules/cm². The time to achieve this kill factor moves from nearly infinity to 1500 seconds in Kowalski's example. Based on the slope of change, using an appropriately high intensity level, t can be made to approach zero and leaving the surviving population orders of magnitude lower.

The chamber design shown in FIG. 40 uses a silicon dioxide lense to expose incoming air to an intensity of 4,000,000 micro joules per cm. sq. At a flow rate of 1.5 liters per second (90 SLM) the UVC light in the chamber (using the k for influenza A of 0.001187 and averaging k2 for the three viruses with measured values for k2 of 2.66 e⁻³) leaves a total surviving population of 8.8 e⁻¹².

Utilizing the multihit model where S=1−(1−e^{-kIt})n for an n of 1.18, where all other variables are the same at a flow rate of 90 SLM, the surviving population S is 9.0 e⁻¹⁴ A one inch diameter SiO₂ lens 4000 having a focal length of 248 mm (>99% transmissivity) is utilized to focus 0.8 watts from a 5 watt (input power) Philips UVC lamp 4002 on an incoming air stream flowing through a 7 mm diameter hole 4004 formed in a SiO2 disk 4006 with an aluminum deposition on its back (lower) side. The 253.7 nm UVC light reflected by an Edmund Scientific SiO₂ lens 4008 with a 0.1 cm Al deposition on the collection (lower) side. The projection of the UVC beam and its back reflection results in an intensity of 4.0 million microjoules/cm² of UVC light being projected on the incoming air.

The lamp region 4010, which is defined by an aluminum reflector 4012, is temperature controlled by averaging the closest 2 of 3 temp sensors 4020 to sweep the temperature range between 35C and 45C and controlling cooling using an in-line cooling fan) to fix the temperature at the level where the UVC output (measured by averaging the closest 2 of 3 UVC photo detectors 4022) is a maximum.

Even though the chamber is designed to sterilize air at a flow rate of 90 SLM the blower will need to be capable of providing 170 SLM. This extra capacity is required because of the wide variation of peak airflow requirements among adults performing light work. In studies performed by a NIOSH contractor, subjects measured blood oxygen saturation levels dropped as low as 92% when positive air pressure respirators (PAPR) systems could not meet peak inhalation demands in higher work/stress studies. While, this is not a catastrophic level for blood oxygen levels, it is good practice for inhalation requirements to be met, and is probably why NIOSH set the minimum at 170 SLM.

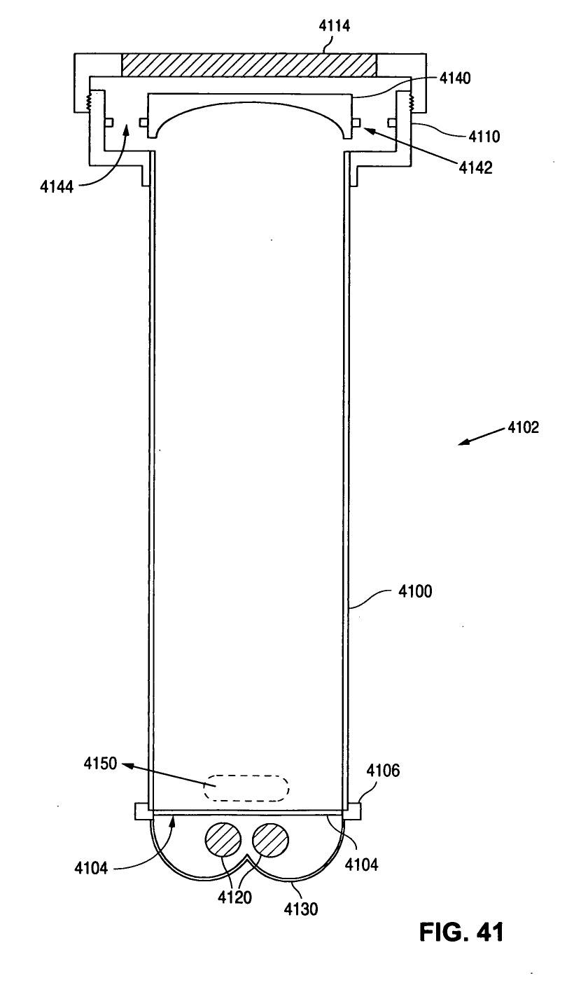

Yet another embodiment of the invention is shown in FIG. 41. In this embodiment the housing 4100 of the kill chamber 102 comprises a glass tube with a reflective coating on its inner surface. In this embodiment the tube has a 2 inch inside diameter but in other embodiments 2.5 inch inside diameter and 3 inch inside diameter tubes were used. The tube 4100 is closed off at its lower end by a titanium doped quartz disk 4104 held in place by means of a GE UV plastic ring or joint 4106. At its upper end, the 4100 is provided with a plastic end cap 4110 having a threaded upper portion that engages complementary threads on a filter cap 4112. The filter cap 4112 includes a HEPA filter 4114 for filtering particles from incoming air. In this embodiment, the UV lamp 4120 is arranged perpendicularly to the longitudinal axis of the housing 4100 and is housed in a reflective housing 4130 for reflecting light upward toward the upper end of the housing. The upper end of the housing, in turn, is provided with a parabolic glass mirror 4140 having an Al/SiO coating on its lower, curved surface. The mirror 4140 is held in place by means of a plastic mirror support 4142 that takes the form of an annular shaped plastic disk with holes 4144 for allowing air to pass from the filter 4114 into the housing 4100. The air moves through the kill chamber 4102, which in this embodiment has a volume of 0.35 liter, and passes out of the housing 4100 through a hole 4150. The hole 4150 leads to a blower which moves the air to a face mask (not shown).

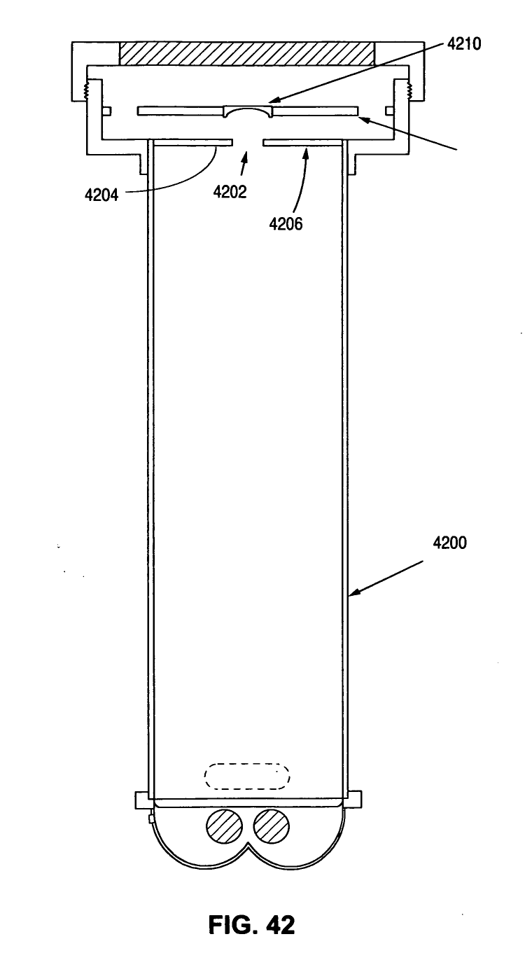

Another embodiment of the invention is shown in FIG. 42 which is similar to that of FIG. 41. However, in this embodiment, the air entering the air flow housing or chamber 4200 is constrained to a small opening 4202 by a quartz disk 4204 with a reflective lower surface 4206. The upper reflector 4210, is correspondingly smaller than that of FIG. 41. The reflector 4210 comprises a glass mirror with reflective coating e.g., aluminum deposition with MgF₂ overcoat. The other elements are substantially the same as in FIG. 41 and are therefore not described again.

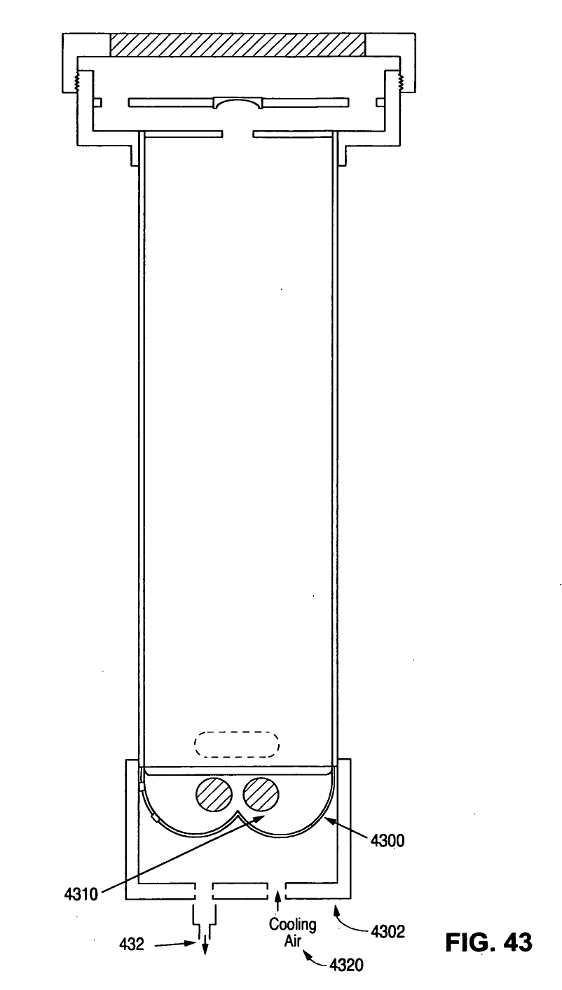

Yet another embodiment is shown in FIG. 43, which is similar to the embodiment of FIG. 42 but in addition to the lamp housing 4300, it includes a further, outer housing 4302 for the UV lamp 4310. The outer housing 4302 has a cooling air input 4320 and an output 4322 to cool the lamp 4310.

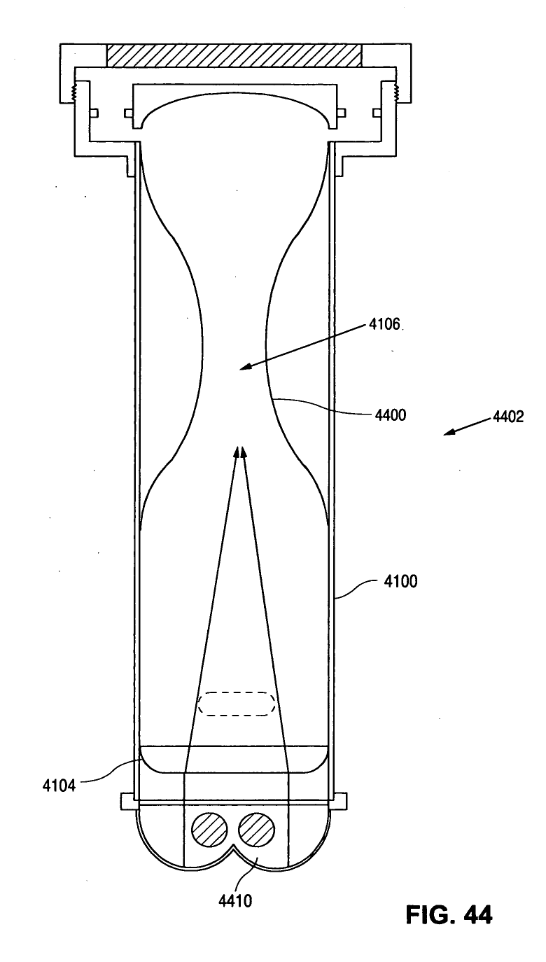

FIG. 44 shows yet another embodiment, which is similar to the embodiment of FIG. 41. Therefore similar elements are not discussed here again. However, this embodiment differs in that an inner hour-glass shaped wall 4400 is provided to constrain the air flow path to a narrow region near the middle of the housing 4402. The UV lens 4104 in this embodiment has its focal point coinciding with the narrow portion 4106 of the hour-glass shaped wall 4400.

The lamp housing in the embodiments of FIGS. 37-44, e.g the housing defined by tube 3702, by 3832, by 4012, by 4130, inner housing 4300, and housing 4410 is kept at a slight negative pressure e.g., 500 torr of dry N₂. This helps protect the user against leakage in the event of UV lamp failure.

What is important in all of the portable devices that feed a face mask, is that energy needs to be conserved to facilitate the use of a portable power supply. Hence the use of a fan or blower rather than a pump to suck the air into the kill chamber and through to the face mask, is preferred. In order to avoid simply having to use a high power blower to provide a large positive pressure in the face mask, the present invention proposes using minimal size blowers while still providing a positive pressure in the mask. Since conventional size HEPA filters used in masks (especially high quality filters such as N100 which are certified to filter out 300 nm particles to 99.97%) have relatively small diameter and produce a large pressure differential, they are not suitable for use with low power blowers. The present invention addresses this issue by providing for larger diameter HEPA filters to be used. In addition power management is achieved by providing constant pressure or constant flow rate. In a preferred embodiment, pressure in the mask is sought to be maintained constant as user inhales and exhales or changes his/her exertion. This means that the blower power is adjusted as needed e.g. by controlling its current based on one or more pressure sensors. In some of the embodiments three pressure sensors were used and the average taken of the two closest values. As discussed above, instead of adjusting power to the blower, the flow can be varied using a valve in the connection between the kill chamber and the mask. To further reduce power consumption, positive pressure in the mask is kept to a very low value, preferably 0.5 to 1 inch of water pressure over ambient pressure. This arrangement allows the use of a few masks that are not necessarily fitted masks but loosely fit the contours of a limited number of generic human faces. In one embodiment, several different generic sizes, e.g., extra small, small, medium, large, extra large masks were provided for attachment to the kill chamber in order to accommodate different users and provide them all with a relatively good fit. For purposes of this invention, the term “loosely fitting” will be used to describe such a mask fit.

The invention further contemplates not only allowing different face masks to be used with a kill chamber by having the face mask and kill chamber connected by a releasable connection, it also contemplates a modular arrangement for the other elements. As such, the invention provides for a separate housing or chamber for the UV lamp and for the air supply. For instance in FIG. 41, the lamp 4120 is mounted in its own chamber defined by reflector 4130. The air supply, in turn is constrained to an air flow housing 4100 which has its air flow inlet at the filter 4114 and air flow outlet at hole 4150 leading to the blower. According to the invention, the air flow housing 4100 and lamp housing 4130 are separably connected. Furthermore, the lamp 4120 the blower or fan that helps move the air through the air chamber, are provided with their own power supplies and electronics to allow the air chamber to work independently of the UV lamp. This allows the system to be used with or without UV sterilization. Also, the modularity of the system allows for the various elements to be separably sold and replaced and facilitates interchangeability of elements by different suppliers, provided common connectors are used. In one embodiment the air flow housing and lamp housing is provided with a charger arranged for simultaneous charging of the batteries for the blower and the batteries for the UV lamp. In particular, the charger plug is provided with two pairs of pins for simultaneous charging of the blower and UV lamp, and provide for the ability to use the same charger if only the blower without the UV lamp is used. In such a case only one of the pairs of pins is engaged for charging the batteries for the blower.

As another feature of the invention, the present embodiment provides for safeguards against ozone production. In order to minimize the production of ozone, a ZrO₂. layer is provided on the quartz plate or the bulb. This reduces the peaks between the wavelengths 185-250 nm. In addition in some embodiments HFlO₂ was also, or instead, sputtered onto the reflective surfaces. Also a titanium sponge was placed in the air flow path in the chamber in some embodiments.

As discussed above, it is desirable to provide a highly reflective coating on the inner surface of the housing of the kill chamber in order to ensure multiple reflections of the UV light and thereby increase the effectiveness of the light in destroying the DNA or RNA of any biological contaminants entering the kill chamber. Various materials and coatings may be used as discussed below. However, some processes for applying coatings are best performed prior to forming the housing since the coating may be such that it is prone to peeling off if the surface on which it is formed is subsequently deformed or bent.

The housing of the kill chamber, in one embodiment, comprises an aluminum pipe in which the inner surface is polished and is then exposed to a chemical vapor deposition (CVD) process to increase its UV reflectivity. This may involve organo-metallic CVD (OMCVD), or plasma enhanced CVD (PECVD).

In another embodiment, instead of CVD, a reflective coating was applied to the inner surface by sputtering on a reflective coating e.g., Al sputtered onto a highly polished surface e.g., several 100 nm thick—preferably 300 nm thick or more.

In yet another method of applying a highly reflective surface the housing material, e.g. in the case of an Al housing material the Al material was placed in a chemical bath and the chemical reacted with the Al in an electroplating process. The aluminum could be electrically connected to act as the anode or the cathode.

In yet another approach to applying the highly reflective coating an electro-chemical deposition was performed, similar to a corrosion process. In this way an oxide layer was formed on the metal surface of the housing material, which in this embodiment was Al, thereby providing an aluminum oxide layer on the Al.

In yet another approach to applying a highly reflective coating molecular beam epitaxy was performed, involving the use of a high vacuum environment and creating a beam of material to form a layer on the substrate material (in this case on the housing material).

In yet another approach to applying a highly reflective coating ion beam implantation was used to implant highly reflective materials. The formation of a highly reflective coating may be followed by steps for adding additional chemicals by ion beam implantation. Additional chemicals may also be added by ion beam implantation where a highly reflective coating has already been applied. For instance, if Al is first sputtered on, additional material for better reflectivity may thereafter be added by ion beam implantation.

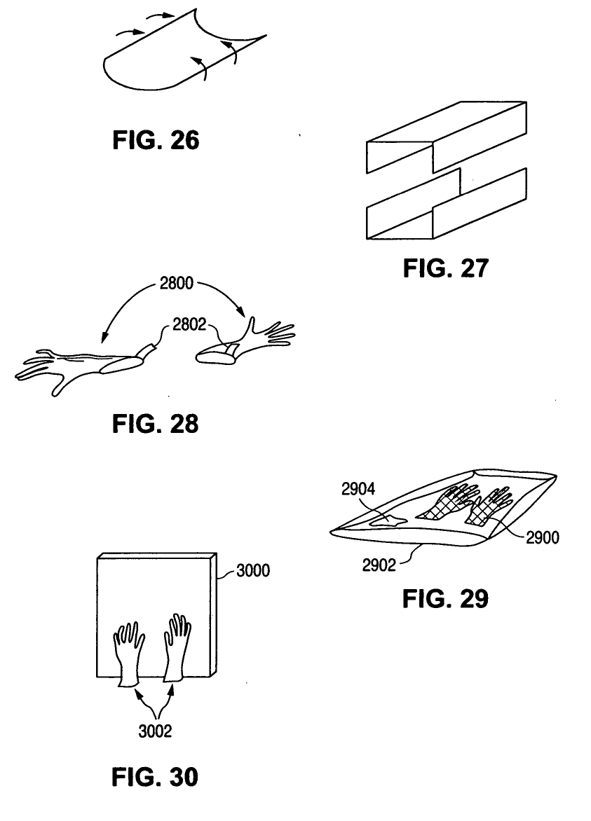

As suggested above, instead of first completing the forming of the housing of the kill chamber or using a tube or pipe, and then adding a reflective coating, the housing may be formed from a flat or open piece of metal that is treated with a reflective coating and preferably also a coating with a high refractive index, prior to being formed (e.g., by bending or folding) into a tube or cylinder (e.g. square or round cylinder) as shown in FIG. 26. The housing may, instead be made from two or more pieces that fit together and may be of any shape that is suitable for forming a housing when the pieces are fitted together, one embodiment of which is shown in FIG. 27. The bent or folded or fitted together pieces may be joined where the edges meet (referred to herein as the joint for convenience) by sealing the joint e.g. by thermal seal such as a thermal weld or using a thermal adhesive or any other suitable technique.

One or more of several materials may be chosen to provide the highly reflective coating. The materials are chosen to preferably have high reflectivity for UVC particularly for UV in range 253-268 nm, e.g., Al alloy e.g. aluminum oxide. Other oxides may also be used e.g. magnesium oxide. The oxides may be applied on their own or after first applying a layer of Al if the chamber material is not itself made of aluminum.

Other compounds such as barium sulphate or alloys (e.g Al alloys or Ag alloys) may be used instead to provide for a highly reflective chamber surface. In yet other embodiments compounds that may chosen from CaF2, MgF2, SrF2, BaF2, LiF, KTiOPO4 (potassium titanyl phosphate or KTP), CaCO3 (calcite), BaB2O4 (beta barium borate or BBB), and HFlO₂, TiO2 were used as highly reflective coatings. In fact multilayers of two or more of these compounds (known as “dielectric stacks”), were used in some embodiments, and in some they were arranged as pairs of layer, e.g. 10 pairs of layers, e.g., pairs such as CaF2 and MgF2 and thicknesses of the various layers adjusted depending on the refractive indices of the materials used. Preferably alternating layers of high- and low-refractive index material are used (where the indices of refraction are at the wavelength for which high reflection is desired). In particular, each layer should be an odd multiple of one-quarter of the wavelength of the light in the medium, i.e. an odd multiple of one-quarter of the vacuum wavelength divided by the index of refraction. Thus, if the indices of refraction of the two materials are 1.433 and 1.762 and the vacuum wavelength is 265 nm, you would want alternating layers with a thickness of 46 nm (or 139 nm, or 231 nm, or other odd multiples) for the low-index material and with a thickness of 38 nm (or 113 nm, or 188 nm, or other odd multiples) for the high-index material.

In yet another embodiment a polymer, in this case Teflon, was sprayed on. Instead the Teflon or other polymer could be applied by dipping the housing material into a Teflon bath before or after forming the housing.

In the case where the housing is cylindrical to start with or in two parts that don't need subsequent bending, one alternative was to provide barium sulphate particles in suspension with UV resistant polymer, which was then sprayed on. Instead it could be applied by dipping.

In one embodiment instead of applying a highly reflective coating, the housing itself was made from a UV resistant polymer containing barium sulphate particles.

In order to protect the user not only while he or she is wearing the apparatus of the invention, but also in the process of taking the apparatus and clothing items off, and to ensure that the apparatus and clothing items are themselves decontaminated after use, the present invention also proposes a methods and means for assisting in removing gloves and decontaminating the various items.

FIG. 28 shows a pair of gloves 2800 with tabs or strips 2802 that allow the user to easily remove the gloves. As an added protection, the tabs 2802 in this embodiment are provided with a biocide coating.

Typically the gloves will be sealed in an air proof package or bag as shown in the embodiment of FIG. 29. To avoid the biocide, which commonly has a crystalline structure, from absorbing moisture prior to removal of the gloves 2900 from their package 2902 the gloves 2900 are stored in the package 2902 with a desiccant 2904. Instead of tabs, the gloves 2900 of this embodiment are made from a woven material impregnated with a biocide. In another embodiment, instead of a woven material or tabs, the entire glove was treated with a biocide coating.

As part of another aspect of the invention, instead of treating the gloves to facilitate removal without contamination, a sticky board 3000 is provided as shown in FIG. 30. The board 3000 is attachable to a wall or other secure surface, and removal of the user's gloves 3002 is achieved by the user touching the board and slipping the hands out of the gloves as the gloves 3002 are retained by the sticky board.

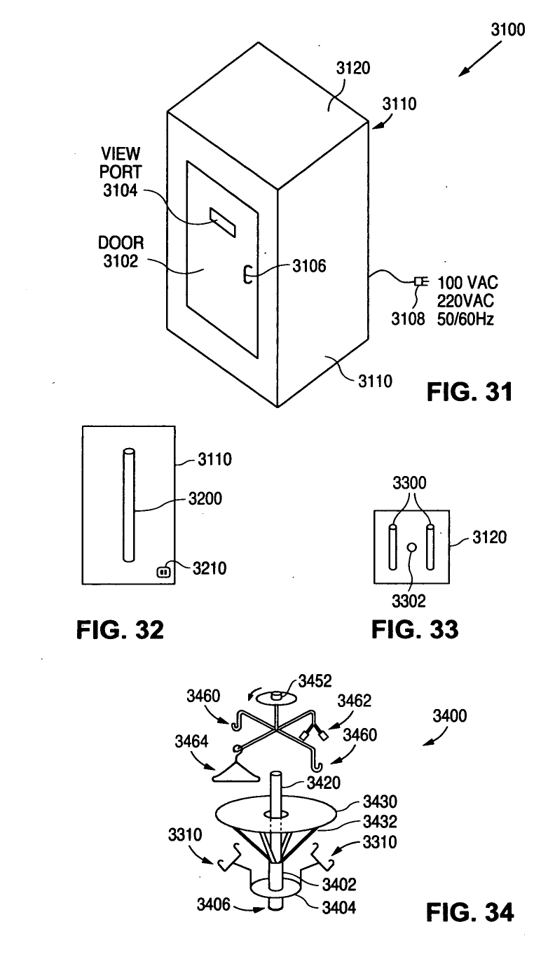

As a further aspect of the invention, there is provided a decontamination chamber and a method of de-contaminating the apparatus of the invention, namely the kill chamber and face mask with goggles, as well as clothing items worn by the user. One embodiment of a decontamination chamber of the invention is shown in FIGS. 31-33. The decontamination chamber 3100 comprises a polished Al chamber, which in this embodiment is coated on the inner wall with paint that contains barium sulphate for greater reflectivity. The decontamination chamber may instead be made of plastic coated with a UV reflective coating, e.g. by sputtering on a highly reflective coating, and can be made in two halves that are then joined, e.g. by a thermal seal or by using adhesive (e.g., a thermal adhesive). Thus the entire discussion of the formation, coatings and materials for the kill chamber apply equally to the formation of the decontamination chamber and are not repeated here for convenience but are understood as applying to the decontamination chamber as well.

The chamber 3100 of this embodiment has a door 3102 with a view port 3104 and handle 3106.

A timer 3107 is also included allowing the decontamination time to be set. The timer may be coupled to a visual or audible indicator such as a buzzer to advise the user of the decontamination chamber when the decontamination time is up. In addition the timer may be connected to the UV light sources to switch the lights off when the defined time is reached. In another embodiment, a timer and locking mechanism may be included in the door (similar to a front loading washing machine) to automatically lock the door for a defined time. In yet another embodiment, where subsequent access to chamber may be desired for further additional loading of material, a preset timer may be included in which the time is simply reset to start over whenever the door is opened.

An electrical access point 3108 extends to an electrical outlet mounted on the inside of the chamber as is discussed in greater detail below.

FIG. 32 shows the inner side of the side panels and back panel 3110 of the chamber 3100 and FIG. 33 shows the upper and lower panels or walls of the chamber 3100. Each panel or wall 3110, 3120 is provided with a UV light source. As shown in FIGS. 32 and 33, the light source on the panels 3110 comprises a mercury vapor lamp 3200, while in the case of the panels 3120 the light source comprises two mercury vapor lamps 3300. In a preferred embodiment the chamber door 3102 is provided with a switch (not shown) that turns off the UV light source when the door opens. This may be a mechanical switch or a UV photo detector with an electric switch. In addition a photodetector is preferably provided for each UV light bulb or, in the case of a UV LED light source, banks of LEDs may be provided with photodetectors to indicate when to change the bulbs or LEDs.

In the case of the panels 3110, the back panel also includes an electrical outlet 3210, which is shown in FIG. 32 and which receives power via the access point 3108 discussed above with respect to FIG. 31. It will be appreciated that while the side panels 3110 may also be provided with electrical outlets, typically the chamber 3100 need only have one electrical outlet for plugging in a battery pack of the kill chamber. It will also be appreciated that the battery pack of the kill chamber may be implemented to be chargeable by magnetic coupling similar to an electric toothbrush, in which case the chamber 3100 may be provided with a complementary charging socket.

FIG. 33 shows that both panels 3120 are in turn provided with recesses 3302 for receiving a rotational assembly, which is illustrated in FIG. 34.

The rotational assembly 3400 of this embodiment includes a support post 3402 mounted on a platform 3404 and has a bearing 3406 mounted on its lower surface, which is receivable in the recess 3302 of the lower panel 3102. Shoe holders or racks 3310 are also mounted to the platform 3404 for supporting the user's shoes. In order to ensure UV radiation from the inside as well, a mercury vapor lamp 3420 is mounted on the support post 3402. In one embodiment a support platform 3430 made of UV transparent material, e.g., quartz, is secured to the support post 3402 by means of arms 3432. The support platform 3430 may be used for supporting items such as the kill chamber assembly and face mask used by the user. In another embodiment a rack 3450 may in addition to the platform 3430 or as an alternative to the platform 3430, be mounted by means of a bearing 3452 to the recess 3302 of the upper panel 3120. As shown in FIG. 34, the rack 3450 is suspended from a rotational platform 3454. In the embodiment shown in FIG. 34, the rack 3450 includes hooks 3460 for the backpack respirator and face mask. It also includes a pants hanger 3462 and jacket hanger 3464. The entire rotational assembly is rotated for maximum exposure to UV radiation from the UV light sources mounted on the side, back, upper and lower panels of the decontamination chamber. In this embodiment a drive motor (not shown) is connected to the platform 3404 and a second drive motor is connected to the platform 3454. It will be appreciated that in other embodiments the rack 3450 could be connected to the mercury vapor lamp 3420 or the support post 3402 by means of brackets, thereby avoiding the need for two drive motors. It will also be appreciated that the mercury vapor lamp 3420 could be replaced with a UV LED or a bank of UV LEDs.

While specific embodiments were discussed above, it will be appreciated that these were included by way of example only and that the present invention is not limited to the embodiments discussed, but includes other embodiments as defined by the scope of the claims. Furthermore, while the above embodiments discussed with respect to FIGS. 1-17, 35 and 36 dealt specifically with a portable sterilization apparatus, the present invention relating to the protection of UV LED light sources through polarized coatings or lenses, the supplementation of mercury vapor lamps and/or protective quartz sleeves around the lamps with titanium by deposition or implantation, and the types of materials and methods of providing a highly UV reflective inner surface to the housing of the chamber, are equally applicable to a non-portable sterilization apparatus for sterilizing not only air but also objects placed in the housing of the apparatus, such as the decontamination of clothing and portable air sterilization apparatus in a decontamination chamber as described with respect to FIGS. 31-34. Similarly, the shaping of the housing for channeling air or inclusion of baffles to channel air close to the UV light source is equally applicable to a non-portable air sterilization apparatus. The claims are accordingly defined to cover all such sterilization apparatus. The relevance of the proposals in the present invention to bring the air into close proximity with the UV source and use highly reflective housing surfaces and hopefully eliminate some of the water vapor in the air through the use of the filtering at the air intake, is borne out by academic studies and models such as that described in the article “Mathematical Modeling of Ultraviolet Germicidal irradiation for Air Disinfection” by W. J. KOWALSKI, W. P. BAHNFLETH, D. L. WITHAM, B. F. SEVERIN, and T. S. WHITTAM (Quantitative Microbiology 2, 24-9270, 2000 # 2002 Kluwer Academic Publishers.

As mentioned above, the use of lenses or other means to create high intensity radiation in order to reduce secondary survival populations of pathogens is applicable also to systems that are not specifically geared to supply individual users with sterilized air. The approach discussed above of dealing with secondary survival rates is applicable also to air sterilizers that serve large groups of people, e.g., air sterilizers used in heating duct systems. It will also be appreciated that all of the embodiments above are given by way of example only and are not intended to limit the invention as defined by the claims.

What is claimed is:

1. An air sterilizer, comprising
a UV light source for providing UV radiation of a first intensity,
a blower for generating an air stream,
a particle filter for filtering particles out of the air stream, and
means for radiating pathogens in the air stream with high intensity UV light in a high intensity zone, wherein the high intensity light is of a higher intensity than the first intensity.

2. An air sterilizer of claim 1, wherein the means for radiating pathogens with a high intensity UV light includes a UV beam magnifier focusing UV light onto the high intensity zone.

3. An air sterilizer of claim 2, wherein the UV beam magnifier comprises a UV lens.

4. An air sterilizer of claim 1, further comprising an air flow housing and means for automatically adjusting air pressure or air flow rate of an air stream passing through the airflow housing to account for changes in the demand.

5. An air sterilizer of claim 3, wherein the high intensity zone comprises a passageway or hole located at the focal point of the lens.

6. An air sterilizer of claim 1, wherein the UV light source is a mercury vapor lamp, at least one LED or a flashlamp.

7. An air sterilizer of claim 1, wherein the particle filter is a HEPA filter.

8. An air sterilizer of claim 1, further comprising an airflow housing with a first and a second input and a face mask having an input and an output, wherein the output of the face mask is connected to the second input of the housing.

9. An air sterilizer of claim 8, wherein the air flow housing is divided into a first section in flow communication with the first input, and a second section in flow communication with the second input.

10. An air sterilizer of claim 1, wherein the UV light source includes a mercury vapor lamp mounted in a lamp housing.

11. An air sterilizer of claim 1, wherein the high intensity zone includes a reflector for reflecting back UV light.

12. An air sterilizer of claim 4, wherein the air flow housing includes a UV reflective surface provided with a coating for reducing ozone.

13. An air sterilizer of claim 12, wherein the coating for reducing ozone includes HFlO₂.

14. An air sterilizer of claim 10, wherein the air flow housing and lamp housing include transparent walls, and at least some of the transparent walls are provided with Ti or ZrO₂.

15. An air sterilizer of claim 1, further comprising a Ti sponge mounted in the air stream.

16. An air sterilizer of claim 10, wherein the air flow housing houses a blower or fan, and is separably connected to the lamp housing.

17. An air sterilizer of claim 16, wherein the blower or fan, and the mercury vapor lamp include their own power supplies.

18. An air sterilizer, comprising
an air flow housing having first and second inputs for receiving air from two different air source, and an output,
a UV light source,
a fan or blower for moving air through the air flow housing, and
a particle filter for filtering air entering through at least the first input.

19. An air sterilizer of claim 18, wherein one air source is the surrounding air and the other air source is exhaled air fed into the housing from a face mask.

20. An air sterilizer of claim 18, further comprising a UV beam magnifier for focusing the UV light source on a defined region.

21. An air sterilizer of claim 20, wherein the defined region includes a reflector for reflecting back UV light.

22. An air sterilizer of claim 18, wherein the air flow housing includes a UV reflective surface provided with a coating for reducing ozone.

23. An air sterilizer of claim 18, further comprising a light source housing.

24. An air sterilizer of claim 18, wherein the blower and light source are provided with their own power supplies and their own charging ports.

25. An air sterilizer of claim 23, wherein the air flow housing houses a blower or fan, and is separably connected to the light source housing.

26. An air sterilizer of claim 23, wherein the air flow housing and light source housing include transparent walls, and at least some of the transparent walls are provided with Ti or ZrO2.

27. An air sterilizer of claim 18, further comprising a titanium sponge.

28. An air sterilization system for providing a sterile air supply to a face mask, comprising
a face mask having an air input and an air output,
a kill chamber that includes an air flow housing having a first input for receiving air from the surrounding air, and an output connected to the air input of the face mask,
a UV light source,
a fan or blower for generating an air stream through the air flow housing,
a particle filter for filtering incoming air from the surrounding air, wherein the system includes one or both of a UV beam magnifier and a second input to the air flow housing connected to the output from the face mask.

29. A system of claim 28, wherein the particle filter is a HEPA filter mounted in or on the air flow housing.

30. A system of claim 28, further comprising means for automatically adjusting air pressure in or air flow through the face mask to account for changes in the demand.

31. A system of claim 30, wherein the means for automatically adjusting includes a sensor for measuring the flow rate or the air stream or air pressure, the sensor signal being used to control the flow rate of said air stream or the air pressure in the face mask.

32. A system of claim 31, further comprising an electrically controlled valve mounted in the air flow housing, wherein the flow rate or pressure is controlled by controlling power to the fan or blower or by adjusting the valve, or by controlling both the fan or blower, as well as the valve.

33. A system of claim 31, wherein the sensor is a pressure sensor and signals from the pressure sensor are used for maintaining a constant pressure.

34. A system of claim 33, wherein the constant pressure is maintained by controlling at least one of an electrically controlled valve and current to the blower or fan.

35. A system of claim 34, wherein the electrically controlled valve includes a latch.

36. A system of claim 28, wherein the first input to the housing is a 0.5 to 1 cm diameter hole.

37. A system of claim 28, wherein the UV light source is a flash lamp producing a high intensity burst of UV light.

38. A system of claim 28, wherein the beam magnifier is a UV lens.

39. A system of claim 28, further comprising a light source housing for the UV light source.

40. A system of claim 39, wherein the air flow housing and light source housing are separably connected.

41. A system of claim 40, wherein the fan or blower, and the UV light source are provided with their own portable power supplies.

42. A system of claim 39, wherein the air flow housing and light source housing include UV reflective surfaces provided with a coating for reducing ozone.

43. A system of claim 39, wherein the air flow housing and light source housing include transparent walls, and at least some of the transparent walls are provided with Ti or ZrO₂.

44. A method of reducing pathogens in an air stream comprising,
exposing the air stream to at least two different UV radiation intensities at different periods of time.

45. A method of claim 44, wherein the at least two different UV radiation intensities are provided by a UV lamp and a beam magnifier.

46. A method of claim 45, wherein the beam magnifier is a UV lens.

47. A method of claim 46, wherein the beam magnifier focuses the UV radiation onto a channel or hole through which the air stream is forced to pass.

48. A method of claim 44, wherein the radiation exposure is increased by providing reflectors for reflecting the UV radiation.

49. A method of claim 45, wherein the efficiency of the UV light source is enhanced by controlling the temperature of the UV light source.

50. A method of providing protection against air borne pathogens, comprising
filtering the air using a particle filter,
providing a loosely fitting face mask for channeling the filtered air to a user,
providing a fan or blower for generating an air stream, and
controlling the air pressure in the face mask to maintain substantially a constant pressure, slightly positive pressure as demand changes.

51. A method of claim 50, wherein the slightly positive pressure is 0.5 to 1 inch of water pressure over ambient pressure.

52. A method of claim 50, further comprising sterilizing the air channeled to the user.

53. A method of claim 52, further comprising sterilizing the air exhaled by the user.

54. A method of claim 50, wherein the air pressure is controlled by controlling at least one of a flapper valve and the fan or blower.
